(19) United States
(12) Patent Application Publication
(10) Pub. No.: US 2015/0258352 A1
(43) Pub. Date: Sep. 17, 2015

(54) FREQUENCY COMPOUNDING ULTRASOUND PULSES FOR IMAGING AND THERAPY

(71) Applicants: Kuang-Wei LIN, Ann Arbor, MI (US); Charles A. CAIN, Ann Arbor, MI (US)

(72) Inventors: Kuang-Wei LIN, Ann Arbor, MI (US); Charles A. CAIN, Ann Arbor, MI (US)

(21) Appl. No.: 14/656,633

(22) Filed: Mar. 12, 2015

Related U.S. Application Data
(60) Provisional application No. 61/952,031, filed on Mar. 12, 2014.
(60) Provisional application No. 61/952,035, filed on Mar. 12, 2014.

(51) Int. Cl. A61N 7/00; A61B 17/22

(52) U.S. Cl. CPC A61N 7/00; A61B 17/22004; A61N 2007/0073; A61N 2007/0078; A61N 2007/0052; A61N 2007/0039; A61B 2017/22008

(57) ABSTRACT

Methods and devices for producing cavitation in tissue are provided. In one embodiment, a low-frequency ultrasound pulse is transmitted into tissue, a high-frequency ultrasound pulse is transmitted into tissue, and a composite waveform is formed in the tissue that has a peak negative pressure value that exceeds an intrinsic threshold for cavitation in the tissue. In some embodiments, the peak negative pressures of the individual ultrasound pulses do not exceed the intrinsic threshold for cavitation. In another embodiment, a plurality of ultrasound pulses at various resonant frequencies are transmitted into tissue, and the time delays between these transmissions are adjusted to allow the ultrasound pulses to align temporally and form a monopolar pulse having a compound peak negative pressure that exceeds an intrinsic threshold for cavitation in the tissue. Systems for performing Histotripsy therapy are also discussed.

### CROSS REFERENCE TO RELATED APPLICATIONS

This application claims the benefit under 35 U.S.C. 119 of U.S. Provisional Patent Application No. 61/952,031, filed Mar. 12, 2014, titled “Dual-Beam Histotripsy”, and U.S. Provisional Patent Application No. 61/952,035, filed Mar. 12, 2014, titled “Frequency Compounding Ultrasound Pulses for Imaging and Therapy”, both of which are incorporated herein by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to treating tissue with cavitation created by ultrasound therapy.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) bubble clouds appear bright on ultrasound imaging thereby confirming correct targeting and localization of treatment; 3) treated tissue appears darker (hypoechoic) on ultrasound imaging, so that the operator knows what has been treated; and 4) Histotripsy produces lesions in a controlled and precise manner. It is important to emphasize that unlike microwave, radiofrequency, or high-intensity focused ultrasound (HIFU), Histotripsy is not a thermal modality.

### SUMMARY OF THE DISCLOSURE

Histotripsy produces tissue fractionation through dense energetic bubble clouds generated by short, high-pressure, ultrasound pulses. When using pulses shorter than 2 cycles, the generation of these energetic bubble clouds only depends on where the peak negative pressure (P−) exceeds an intrinsic threshold for inducing cavitation in a medium (typically 26-30 MPa in soft tissue with high water content). This disclosure provides a strategic method for precise lesion generation in which a low-frequency pump pulse is applied to enable a sub-threshold high-frequency probe pulse to exceed the intrinsic threshold. This pump-probe method of controlling a supra-threshold volume can be referred to herein as “dual beam Histotripsy” or “parametric Histotripsy.”

In one embodiment, a multi-element dual-frequency transducer array can be used to generate parametric Histotripsy pulses. The transducer array can be configured to generate ultrasound pulses at two different frequencies, such as a low-frequency (i.e., 500 kHz) pulse and a high-frequency (i.e., 3 MHz) pulse. When sub-intrinsic threshold “pump”, or low-frequency and “probe”, or high-frequency pulses are applied together, dense bubble clouds (and resulting lesions) can be generated when their peak negative pressures combine constructively to exceed the intrinsic threshold in the target medium (e.g., in tissue). The smallest reproducible lesion cab be varied by adjusting the relative amplitude between the pump and probe pulses, and, with a higher proportion of the probe pulse, smaller lesions can be generated. When the propagation direction of the probe pulse relative to the pump pulse is altered, the shape of the produced lesion can be changed based on the region that exceeds intrinsic threshold. Since the low-frequency pump pulse is more immune to attenuation and aberrations, and the high-frequency probe pulse can provide precision in lesion formation, this parametric Histotripsy approach can be very useful in situations where precise lesion formation is required through a highly attenuative and aberrative medium, such as transcranial therapy or ablation therapy deep in tissue. This is particularly true if a smaller low-attenuation acoustic window is available for the high frequency probe transducer.

This disclosure also provides a frequency compounding technique to synthesize “nearly monopolar” (“half-cycle”) ultrasound pulses. More specifically, these pulses can be generated using a multi-element transducer at various resonant frequencies (e.g., 0.5, 1, 1.5, 2, and 3 MHz). Each frequency component of the transducer can be capable of generating 1.5-cycle pulses with only one high amplitude negative half-cycle. By varying time delays of individual frequency components to allow their principal peak negative peaks to arrive at the focus of the transducer constructively, destructive interference occurs elsewhere in time and space, resulting in a monopolar pulse approximation with a dominant negative phase.

Inverting the excitation pulses to individual elements can result in generation of monopolar pulses with a dominant positive phase. Monopolar pulses with a dominant negative phase are able to produce very precise Histotripsy-type lesions where the peak negative pressure of the compound waveform exceeds the intrinsic threshold in the target tissue. Monopolar pulses with a dominant negative phase can inhibit shock scattering during Histotripsy, leading to more predictable lesion formation using the intrinsic threshold mechanism, while greatly reducing any constructive interference, and potential “hot-spots,” elsewhere. Moreover, these monopolar pulses can have many potential benefits in ultrasound imaging, including axial resolution improvement, speckle reduction, and contrast enhancement in pulse inversion imaging.

A method of providing ultrasound therapy to tissue is provided, comprising the steps of transmitting a low-frequency ultrasound pulse into tissue, and transmitting a high-frequency ultrasound pulse into tissue to form a composite waveform between the low-frequency ultrasound pulse and the high-frequency ultrasound pulse in the tissue, wherein a peak negative pressure value of the composite waveform exceeds an intrinsic threshold for cavitation in the tissue.

Another method of providing ultrasound therapy to tissue is also provided, comprising the steps of transmitting a low-frequency ultrasound pulse into tissue, wherein the low-frequency ultrasound pulse has a first peak negative pressure below an intrinsic threshold for cavitation in the tissue, and transmitting a high-frequency ultrasound pulse into tissue to form a composite waveform between the low-frequency ultrasound pulse and the high-frequency ultrasound pulse, wherein the high-frequency ultrasound pulse has a second peak negative pressure below the intrinsic threshold for cavitation in the tissue, wherein a composite peak negative pressure of the composite waveform exceeds the intrinsic threshold for cavitation in the tissue.

In some embodiments, the high-frequency ultrasound pulse is transmitted at a time delay with respect to the low-frequency ultrasound pulse to allow for a maximum peak negative pressure value to be achieved with the composite waveform.

In other embodiments, the low-frequency ultrasound pulse ranges from 100 kHz and 1 MHz and the high-frequency ultrasound pulse ranges from 2 MHz to 10 MHz.

In some embodiments, the low-frequency ultrasound pulse and the high-frequency ultrasound pulse are transmitted with a single transducer.

In one embodiment, the low-frequency ultrasound pulse and the high-frequency ultrasound pulse are transmitted with more than one transducer.

In some embodiments, the method further comprises adjusting a proportion of an amplitude of the low-frequency ultrasound pulse relative to an amplitude of the high-frequency ultrasound pulse to adjust a size of a lesion created in the tissue. In one embodiment, increasing the proportion of the amplitude of the high-frequency ultrasound pulses decreases a size of the lesion.

In some embodiments, the low-frequency ultrasound pulse and the high-frequency ultrasound pulse are transmitted simultaneously and combine constructively to form the composite waveform.

In one embodiment, the time delay comprises 0 μs such that the peak negative pressure value comprises a maximum peak negative pressure value.

In some embodiments, the ultrasound pulses comprise short (<20 μsec), high pressure (peak negative pressure >10 MPa) shockwave ultrasound pulses at a low duty cycle, typically <5%, minimizing thermal effects.

In one embodiment, the composite waveform creates cavitation in the tissue to form a lesion in the tissue.

In another embodiment, the transmitting the low-frequency ultrasound pulse step further comprises transmitting the low-frequency ultrasound pulse with an electronic controller coupled to an ultrasound transducer. In further embodiments, the transmitting the high-frequency ultrasound pulse step further comprises transmitting the high-frequency ultrasound pulse with an electronic controller coupled to an ultrasound transducer.

A method of providing ultrasound therapy to tissue is further provided, comprising the steps of transmitting a first ultrasound pulse at a first frequency into tissue, transmitting a second ultrasound pulse at a second frequency into tissue, transmitting a third ultrasound pulse at a third frequency into tissue, wherein the first, second, and third ultrasound pulses combine to form a composite waveform in the tissue, wherein a time delay between the first, second, and third ultrasound pulses causes a peak negative pressure value of the composite waveform to exceed an intrinsic threshold for cavitation in the tissue.

In some embodiments, the ultrasound pulses are transmitted with a single transducer.

In other embodiments, the ultrasound pulses are transmitted with a multi-element transducer.

In some embodiments, the method further comprises adjusting a proportion of an amplitude of the first, second, and third ultrasound pulses to adjust a size of a lesion created in the tissue.

In one embodiment, the first, second, and third ultrasound pulses are transmitted simultaneously and combine constructively to form the composite waveform.

In some embodiments, the time delay comprises 0 μs, and wherein the peak negative pressure value comprises a maximum peak negative pressure value.

In one embodiment, the ultrasound pulses comprise short (<20 μsec), high pressure (peak negative pressure >10 MPa) shockwave ultrasound pulses at a low duty cycle, typically <5%, minimizing thermal effects.

A method of providing ultrasound therapy to tissue is provided, comprising the steps of transmitting a plurality of ultrasound pulses at a plurality of resonant frequencies into tissue, and adjusting time delays between transmission of each of the plurality of ultrasound pulses to allow peak negative pressures of the plurality of ultrasound pulses to align temporally and form a monopolar pulse having a compound peak negative pressure that exceeds an intrinsic threshold for cavitation in the tissue.

In one embodiment, the method further comprises creating cavitation in the tissue with the monopolar pulse to form a lesion in the tissue.

In some embodiments, the plurality of resonant frequencies range from 100 kHz to 10 MHz.

A Histotripsy therapy system is also provided, comprising a pulse generator, an ultrasound therapy transducer coupled to the pulse generator and having a plurality of transducer elements, wherein one or more transducer elements of the ultrasound therapy transducer are configured to transmit a low-frequency ultrasound pulse into tissue that has a first peak negative pressure below an intrinsic threshold for cavitation in the tissue, and wherein one or more transducer element of the ultrasound therapy transducer are configured to transmit a high-frequency ultrasound pulse into tissue that has a second peak negative pressure below the intrinsic threshold for cavitation in the tissue, and an electronic controller coupled to the pulse generator and the ultrasound therapy transducer, the electronic controller configured to control transmission of the high-frequency ultrasound pulse relative to transmission of the low-frequency ultrasound pulse to form a composite waveform between the low-frequency ultrasound pulse and the high-frequency ultrasound pulse that has a composite peak negative pressure that exceeds the intrinsic threshold for cavitation in the tissue.

A Histotripsy therapy system is provided, comprising a pulse generator, an ultrasound therapy transducer coupled to the pulse generator and having a plurality of transducer elements, wherein one or more transducer elements of the ultrasound therapy transducer are configured to transmit a plurality of ultrasound pulses into tissue at a plurality of resonant frequencies, and an electronic controller coupled to the pulse generator and the ultrasound therapy transducer, the electronic controller configured to adjust time delays between transmission of each of the plurality of ultrasound pulses to allow peak negative pressures of the plurality of ultrasound pulses to align temporally and form a monopolar pulse having a compound peak negative pressure that exceeds an intrinsic threshold for cavitation in the tissue.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a Histotripsy therapy system.

FIGS. 2(a)-(e) show calibration results of the 500 kHz component (a total of 12 elements) in the dual-frequency array transducer.

FIGS. 3(a)-(e) show calibration results of the 3 MHz component (a total of 7 elements used, without the top element) in the dual frequency transducer.

FIG. 4 shows results for the experimental set that varied the time delay between the low-frequency “pump” (500 kHz) and high-frequency “probe” (3 MHz) pulses.

FIG. 5 illustrates quantitative results for the experimental set that varied the time delay between pump (500 kHz) and probe (3 MHz).

FIG. 6 gives results for the experimental set that varied the relative amplitude between the probe (3 MHz) and pump (500 kHz) pulses.

FIG. 7 shows quantitative results for the experimental set that varied the relative amplitude between the probe (3 MHz) and pump (500 kHz) pulses.

FIG. 8 shows the number of incidental bubbles generated in the periphery of the focus during the experimental set that varied the relative amplitude between the low-frequency (3 MHz) and high-frequency (500 kHz) pulses.

FIG. 9 shows results for the experimental set that varied the propagation direction of the low-frequency (3 MHz) pulse relative to the high-frequency (500 kHz) pulse.

FIG. 10 gives quantitative results for the experimental set that varied the propagation direction of the low-frequency (3 MHz) pulse relative to the high-frequency (500 kHz) pulse.

FIG. 11 illustrates the results for excised porcine hepatic tissue treatment.

FIG. 12 shows representative temporal focal waveforms for individual frequency components of the frequency-compounding transducer.

FIG. 13 shows representative waveforms of a frequency-compounded pulse with a dominant negative phase (a “negative-polarity” pulse).

FIG. 14 displays representative 2D spatial pressure fields for a negative-polarity pulse.

FIG. 15 shows representative temporal focal waveforms for individual frequency components when driving signals are inverted.

FIG. 16 shows representative waveforms of a frequency-compounded pulse with a dominant positive phase (a “positive-polarity” pulse).

FIG. 17(a) shows the ratio of P− to P+ as a function P− in the negative-polarity pulse case. FIG. 17(b) is a representative temporal waveforms for the negative-polarity pulse at P−=20 MPa, measured by the FOPH in free-field. FIG. 17(c) is the ratio of P+ to P− as a function P+ in the positive-polarity pulse case. FIG. 17(d) is a representative temporal waveform for the positive-polarity pulse at P+=23 MPa, measured by the FOPH in free-field.

FIG. 18 shows representative lesion and bubble cloud images in the RBC phantom experiments that used negative-polarity pulses.

FIGS. 19(a)-(b) are quantitative results for the RBC phantom experiments that used negative-polarity pulses.

FIG. 20 shows representative lesion and bubble cloud images in the RBC phantom experiments that used positive-polarity pulses.

FIGS. 21(a)-(d) are example waveforms that could be synthesized by the current frequency-compounding transducer.

FIG. 22 illustrates one method of providing ultrasound therapy to tissue.

FIG. 23 illustrates another method of providing ultrasound therapy to tissue.

FIG. 24 illustrates another method of providing ultrasound therapy to tissue.

FIG. 25 illustrates yet another method of providing ultrasound therapy to tissue.

### DETAILED DESCRIPTION

Histotripsy is a noninvasive, cavitation-based therapy that uses very short, high-pressure ultrasound pulses to generate a dense, energetic, lesion-producing bubble cloud. This Histotripsy treatment can create controlled tissue erosion when it is targeted at a fluid-tissue interface and well-demarcated tissue fractionation when it is targeted within bulk tissue. Additionally, Histotripsy has been shown to be capable of fragmenting model kidney stones using surface erosion that is mechanistically distinct from conventional shockwave lithotripsy (SWL). Histotripsy therapy can be guided and monitored using ultrasound B-mode imaging in real-time, since 1) the cavitating bubble cloud appears as a temporally changing hyperechoic region in B-mode imaging, allowing the treatment to be precisely targeted, and 2) the echogenicity of the targeted region decreases as the degree of tissue fractionation increases, which can be used as a way of monitoring lesion production (image feedback) in real-time.

Generally in Histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This has been called the “shock scattering mechanism”. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rare factional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without these intense shock fronts developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on one or two negative half cycle(s) of the applied ultrasound pulses exceeding an “intrinsic threshold” of the medium (the “intrinsic threshold mechanism”). This threshold can be in the range of 26-30 MPa for soft tissues with high water content, such as tissues in the human body. Using this intrinsic threshold mechanism, the spatial extent of the lesion is well-defined and more predictable. With peak negative pressures (P−) not significantly higher than this threshold, sub-wavelength reproducible lesions as small as half of the −6 dB beamwidth of a transducer can be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). In this disclosure, a strategic application of Histotripsy pulses is described to address this issue: a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach can be referred to herein as “dual beam Histotripsy” or “parametric Histotripsy.”

Parametric Histotripsy uses pulses typically less than two cycles, and the arrival times of the “pump” and “probe” pulses can be adjusted to enhance peak negative pressure P− constructive interference. Additionally, parametric Histotripsy generates a dense bubble cloud without any pre-existing microbubbles or special interfaces.

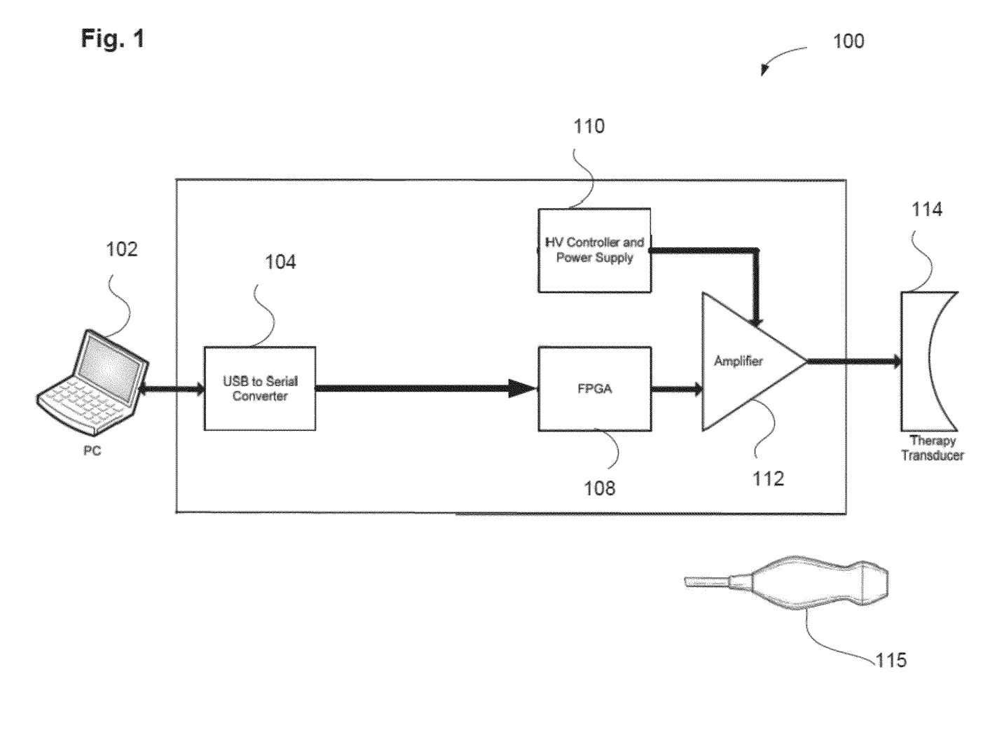

FIG. 1 illustrates a Histotripsy system configured to generate cavitation bubbles or bubble clouds in tissue according to the methods and embodiments described herein. A Histotripsy system and generator is configured to generate complex waveforms in order to support the ultrasound pulse sequences described herein. A simplified block diagram of system 100 is shown in FIG. 1. The main components of the system are: Computer/controller 102, USB to Serial Converter 104, FPGA (Field Programmable Gate Array) 108, High Voltage Controller and Power Supply 110, Amplifier 112, and Therapy Transducer 114.

All controls for the generator can be established using a “Histotripsy Service Tool” software that can run on the computer/controller 102 (e.g., a standard PC, laptop, tablet, or other electronic computing system) and communicates to the generator via a connector such as USB serial communication 104. Therapy, including bubble cloud cavitation and tissue ablation can be imaged and tracked in real time using an imaging system 115, such as an ultrasound imaging system or probe.

The system 100 can be configured to receive multiple sets of different driving parameters and loop them, which give the ability to the user to create wide range of custom sequences where all parameters (pulse repetition frequency (PRF), voltage amplitude, number of cycles, number of pulses per set, frequency, transducer element channels enabled, and time delays) can be set differently for every pulse generated. Time delays between pulses can be specified by the PRF for a parameter set or by specifying zero as the number of cycles per pulse.

For overall voltage amplitude regulation, level of high voltage can be changed accordingly through the HV Controller 110. This method cannot be used for dynamic voltage amplitude changes between two pulses since it will take too long for all capacitors on the HV line to discharge. For dynamic voltage amplitude changes between pulses, PWM (pulse width modulation) can be used at the FPGA 108 where the duty cycle of the pulse may be modulated in order to produce the desired pulse voltage and resultant pressure amplitude.

### Histotripsy Service Tool and Electronic Controller

Histotripsy Service Tool is an application that can be run on any PC or computing system (e.g., electronic controller) and may be used for controlling the system. The Histotripsy Service Tool can start/stop the therapy, set and read the level of high voltage, therapy parameters (PRF, number of cycles, duty ratio, channel enabled and delay, etc.), and set and read other service and maintenance related items. The Histotripsy Service tool and Electronic Controller can be configured to set/read working parameters, start/stop the therapy, etc. It can use internal flash memory or other electronic storage media to store all the parameters. The Histotripsy Service Tool and Electronic Controller can communicate to the FPGA 108 all driving parameters that are necessary to generate complex pulsing. They can also communicate using serial communication or other electronic communication to the high voltage controller and power supply 110 where it can set/read the proper level of driving voltage.

### USB to Serial Converter

USB to Serial converter 104 can convert USB combination to serial in order to communicate from the PC or electronic controller to the FPGA. It should be understood that other converters (or none at all) may be used in embodiments where the connection between the generator and the controller is not a USB connection.

### FPGA

The FPGA 108 receives the information from the PC or electronic controller 102 and it can generate the complex pulsing sequence that is required to drive the amplifier 112. The FPGA can run on 100 MHz clock since speed of pulsing is critical to be timed in at least 10 ns increments.

### High Voltage Controller and Power Supply

The High Voltage Controller and Power Supply 110 determines the level of DC voltage that needs to be supplied to the amplifier circuitry in order to have an adequate voltage amplitude level at the output of the amplifier.

### Amplifier

The Amplifier 112 receives pulses generated by the FPGA and is supplied with high voltage from High Voltage Controller and Power Supply. It generates high voltage amplitude pulses that are fed to the Therapy Transducer 114 through the matching network components which properly matches the impedance of the therapy transducer to the impedance of the amplifier. It can be necessary to use a large number of capacitors that can store enough energy to support peak current demand during the generation of high voltage amplitude pulses.

### Therapy Transducer

The Therapy Transducer 114 can be a single or multi-element ultrasound therapy transducer configured to generate and deliver the ultrasound therapy pulses described herein into tissue or other mediums. In some embodiments, the multi-element ultrasound therapy transducer can generate ultrasound pulses in two or more frequencies. The active transducer elements of the Therapy Transducer can be piezoelectric transducer elements. In some embodiments, the transducer elements can be mounted to an acoustic lens with a common geometric focus.

### Imaging System

The system can include an imaging system 115, such as an ultrasound imaging system, to monitor therapy and track cavitation and tissue ablation in real time. The cavitating bubble cloud generated according to the methods and system of this disclosure can appear as a temporally changing hyperechoic region in B-mode imaging, allowing the treatment to be precisely targeted.

A number of experiments were performed to test the results of parametric Histotripsy. In these experiments, an ultrasound therapy system similar to the one described above, the system comprising a 20-element dual-frequency array transducer, in which low-frequency 500 kHz (pump) and high-frequency 3 MHz (probe) elements were confocally aligned, was used to generate parametric Histotripsy pulses at a pulse repetition frequency (PRF) of approximately 1 Hz. Experimental sets were performed in RBC phantoms wherein: 1) the arrival times of 500 kHz and 3 MHz pulses (both below intrinsic threshold) were varied in order to investigate whether lesions could only be generated when combined peak negative P− values exceeded the intrinsic threshold of the phantom (or tissue), 2) the relative amplitude of 500 kHz and 3 MHz pulses was varied to study the size of the smallest reproducible lesion with different proportions of pump (low-frequency) and probe (high-frequency) pulses, and 3) the relative propagation direction between 500 kHz and 3 MHz pulses was varied to determine the effect on the shape and size of the produced lesions. Finally, selected parametric Histotripsy pulses were tested to validate the results in real tissue.

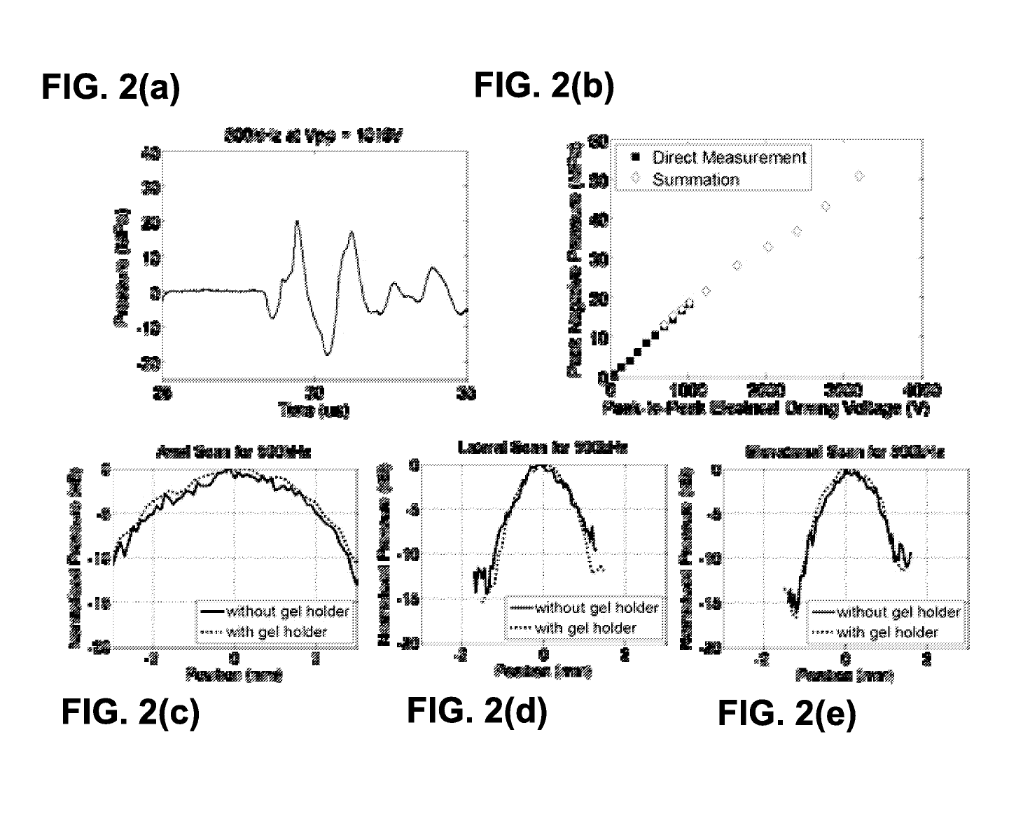

During experimentation, an ultrasonic sensor, such as a fiber-optic probe hydrophone (FOPH) was used to measure the acoustic output pressure of the ultrasound therapy system. FIGS. 2(a)-(e) show the calibration results of the low-frequency (500 kHz) component (a total of 12 elements) in the dual-frequency transducer. FIG. 2(a) shows a typical acoustic waveform of the 500 kHz component at the focus of the transducer before inducing cavitation on the hydrophone tip. Higher pressure levels were estimated by the summation of the output focal P−'s from individual elements. FIG. 2(b) plots the focal acoustic pressure of the 500 kHz component as a function of the peak-to-peak electrical driving voltage to one of the representative elements. The P− estimates from the summation showed a similar trend as the ones from the direct measurement, and they had a good agreement in the range of 700 to 1000 Volts (electrical driving voltage). This peak-to-peak driving voltage is the driving voltage to one representative 500 kHz element. Solid squares (▪) represent pressures from direct measurements with all 12 elements firing at the same time, while empty diamonds (⋄) represent the estimates from the summation of the individual elements firing separately. FIGS. 2(c), 2(d), and 2(e) plot the one-dimensional (1D) beam profiles of the low-frequency (500 kHz) component in the axial, lateral, and elevational directions, respectively. FIG. 2(c) shows a 1D beam profile in the axial direction with (dotted line) and without (solid line) the gel holder. FIG. 2(d) is a 1D beam profile in the lateral direction with (dotted line) and without (solid line) the gel holder. FIG. 2(e) illustrates a 1D beam profile in the elevational direction with (dotted line) and without (solid line) the gel holder. The 6 dB beam-widths in free-field (calculated based on P−) were measured to be 4.89, 1.74 and 1.77 mm in the axial, lateral and elevational directions, respectively.

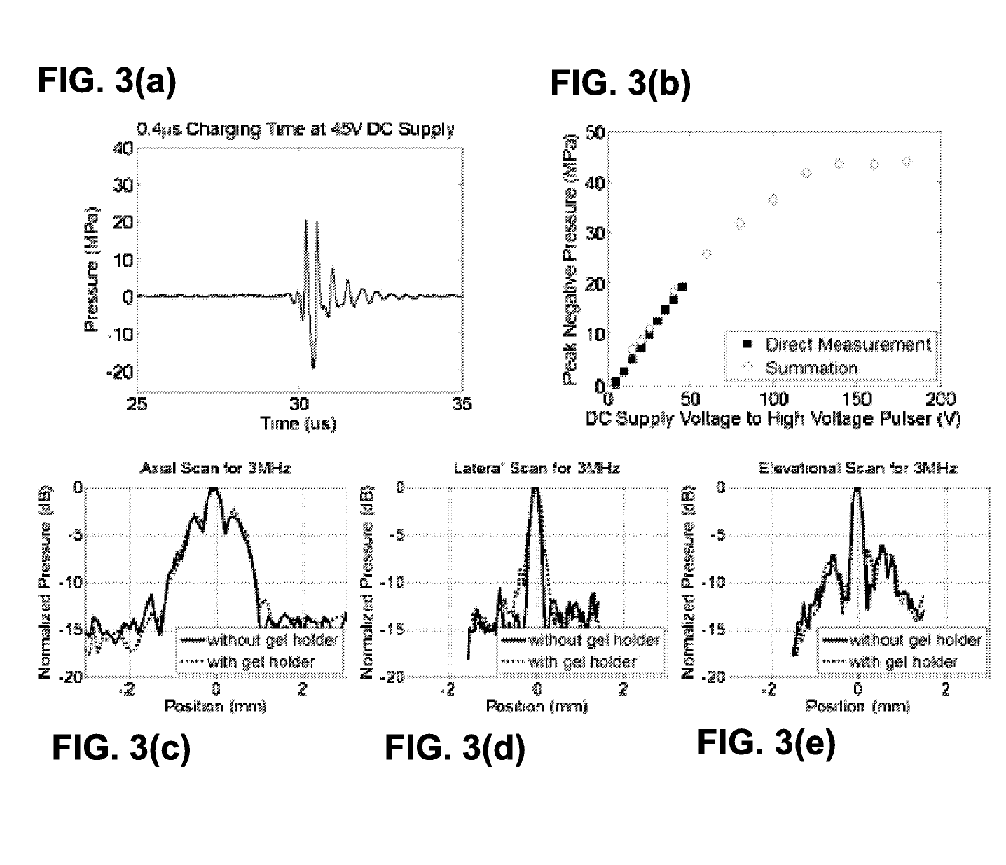

FIGS. 3(a)-(e) show the calibration results of the high-frequency (3 MHz) component in the dual-frequency array transducer during experimentation. FIG. 3(a) shows a typical acoustic waveform of the 3 MHz component at the focus of the transducer before inducing cavitation on the hydrophone tip. FIG. 3(b) plots the focal acoustic pressure of the 3 MHz component as a function of DC supply voltage to the high voltage pulser. The “direct” peak-to-peak electrical driving voltage to the 3 MHz elements was not able to be measured since one of the components in the high voltage pulser was embedded within the housing of the 3 MHz element. Similar to the 500 kHz component, the P−'s after reaching cavitation were estimated by the summation of the output focal P−'s from individual elements, and this had a good agreement with direct measurement in the range of 15 to 45 volts (DC supply voltage to the high voltage pulser). Solid squares (▪) represent pressures from direct measurements with all 7 elements firing at the same time, while empty diamonds (⋄) represent the estimates from the summation of the individual elements firing separately. FIGS. 3(c), 3(d), and 3(e) plot the 1D beam profiles of the 3 MHz component in the axial, lateral, and elevational directions, respectively. FIG. 3(c) is a 1D beam profile in the axial direction with (dotted line) and without (solid line) the gel holder. FIG. 3(d) is a 1D beam profile in the lateral direction with (dotted line) and without (solid line) the gel holder. FIG. 3(e) is a 1D beam profile in the elevational direction with (dotted line) and without (solid line) the gel holder. The 6 dB beam-widths (calculated based on P−) were measured to be 1.42, 0.31 and 0.31 mm in the axial, lateral and elevational directions, respectively.

Cylindrical, custom-made, plastic gel holders (4 cm in diameter and 8 cm in height) with thin polycarbonate membranes (254 μm thick) glued on their sides, were used to hold the RBC phantoms during testing. The influence of the plastic gel holders on focal P− and 1D beam profiles was also investigated. Based on the calibration with a representative plastic gel holder in place, the P−'s were attenuated by 8.2% (500 kHz) and 9.1% (3 MHz); however, the 1D beam profiles did not change significantly, as shown in FIGS. 2(c)-2(e) and 3(c)-3(e). Note that the attenuations for 500 kHz and 3 MHz did not scale with the frequency, and this was likely due to the difference in incidental angle on the gel holder with current arrangement of the transducer element (3 MHz elements had almost normal incidence while 500 kHz elements had oblique incidence).

The applied pressure levels used in the experiments are listed in Table I and II. The applied P− was corrected by the attenuation contributed by the plastic gel holder using the hydrophone measurement discussed above. Additionally, the applied peak negative pressure P− was further corrected for the attenuation contributed by agarose hydrogel and porcine hepatic specimen, using reported values and assuming linear propagation.

TABLE I
PEAK NEGATIVE PRESSURES AND TIME DELAYS FOR CAPTURING BUBBLE
CLOUD IMAGES USED IN RBC PHANTOM EXPERIMENTS

| | | 500 kHZ | | | 3 MHz | | | Time Delay |
|---|---|---|---|---|---|---|---|---|
| | | | Focal P− | | Focal P− | Focal P− | | For |
| | | Focal P− | with | | in | with | | Capturing |
| | | in Free- | Attenuation | | Free- | Attenuation | | Bubble |
| Experiment | | Field | Correction | Proportion | Field | Correction | Proportion | Cloud |
| al Set | Case | (MPa)* | (MPa) | (%) | (MPa)* | (MPa) | (%) | Images (μs) |
| 1 | all | 11.7 | 10.6 | 38 | 20.3 | 17.1 | 62 | 9 |
| | 1 | 0.0 | 0 | 0 | 33.0 | 27.8 | 100 | 5 |
| | 2 | 11.0 | 9.9 | 35 | 21.7 | 18.2 | 65 | 8 |
| 2 | 3 | 15.8 | 14.3 | 51 | 16.1 | 13.6 | 49 | 9 |
| | 4 | 20.8 | 18.9 | 68 | 10.8 | 9.1 | 32 | 13 |
| | 5 | 29.4 | 26.6 | 100 | 0.0 | 0.0 | 0 | 17 |
| 3 | all | 22.3 | 20.2 | 80 | 6.0 | 5.1 | 20 | 15 |

*The P−'s in Experimental Set 1, 2 (Case 2-4), and 3 were linearly interpolated using the directly measured P−'s for various driving voltages. The P−'s of Case 1 and 5 in Experimental Set 2 were linearly interpolated using the linear summed P−'s for various driving voltages.

TABLE II
PEAK NEGATIVE PRESSURES USED IN EX VIVO PORCINE LIVER EXPERIMENTS

| | 500 kHZ | | | 3 MHz | | |
|---|---|---|---|---|---|---|
| | Focal P− | Focal P− with | | Focal P− | Focal P− with | |
| | in Free- | Attenuation | | in Free- | Attenuation | |
| | Field | Correction | Proportion | Field | Correction | Proportion |
| Case | (MPa)* | (MPa) | (%) | (MPa)* | (MPa) | (%) |
| 1 | 17.6 | 17.0 | 53 | 19.5 | 15.3 | 47 |
| 2 | 25.5 | 24.5 | 72 | 12.4 | 10.7 | 28 |
| 3 | 32.0 | 30.8 | 100 | 0.0 | 0.0 | 0 |

*The P−'s in Case 1 (both frequencies) and 2 (only 3 MHz) were linearly interpolated using the directly measured P−'s for various driving voltages. The P−'s in Case 2 (only 500 kHz) and 3 (only 500 kHz) were linearly interpolated using the linear summed P−'s for various driving voltages.

Three different experimental sets were performed in RBC phantoms to investigate lesion production using parametric Histotripsy. The applied P−'s are listed in Table I. Each intended treatment region was exposed with 100 pulses at a PRF of 1 Hz, and a single-focal-point exposure was performed.

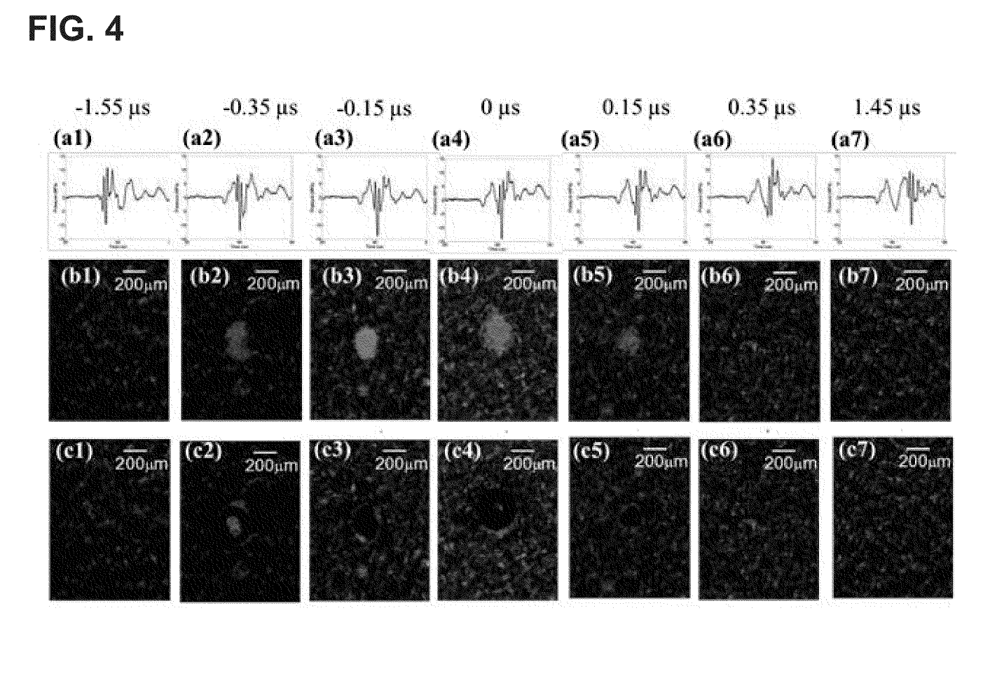

FIG. 4 shows results for an experimental set that varied the time delay between the low-frequency “pump” (500 kHz) and high-frequency “probe” (3 MHz) pulses. The labels on the very top of FIG. 4 indicate the time delay for each column. 0 μs is defined as the time delay that resulted in maximal P− overlap, and negative time delays are defined as the cases when the probe arrives earlier than the pump, and vice versa. Figures in the top row (FIGS. 4(a1)-(a7)) represent the pressure waveforms in free-field for each corresponding time delay. Figures in middle row (FIGS. 4(b1)-(b7)) show representative lesions after 100 Histotripsy pulses for each corresponding time delay. Figures in bottom row (FIGS. 4(c1)-(c7)) show representative bubble clouds or cavitation.

FIG. 4 shows directly measured acoustic waveforms (at lower pressure levels prior to inducing cavitation on FOPH) and representative lesion and bubble images for various time delays. In order to increase readability, only 7 time delays (every other case) are shown. As can be seen from FIG. 4, cavitational bubbles and lesions only occurred when the peak negative pressure P−'s of the 500 kHz and 3 MHz pulses overlapped and added constructively. Neither lesions nor cavitational bubbles occurred where the P−'s of the 500 kHz and 3 MHz signals did not overlap.

The arrival times of the low-frequency (500 kHz) and high-frequency (3 MHz) pulses were varied from the point at which the two did not have any overlap to the point at which they had maximal P− overlap at the focus of the array transducer. More specifically, the time delay for 3 MHz relative to 500 kHz varied from −1.55 to 1.45 μs, where 0 μs indicates the point at which the 500 kHz and 3 MHz pulses had maximal overlap in P−; the negative time delay indicates the P− of the 3 MHz pulse arriving earlier than the P− of the 500 kHz pulse, and vice versa. A total of 13 time delays were investigated, including −1.55, −0.65, −0.35, −0.25, −0.15, −0.05, 0.00, 0.05, 0.15, 0.25, 0.35, 0.65, 1.45 μs. The sample size for each case was nine, leading to a total of 117 lesions generated. The applied pressures were chosen such that each individual frequency component (e.g., the low-frequency component and the high-frequency component) did not individually reach the intrinsic threshold for cavitation; rather, it could be exceeded only by the combination of the two.

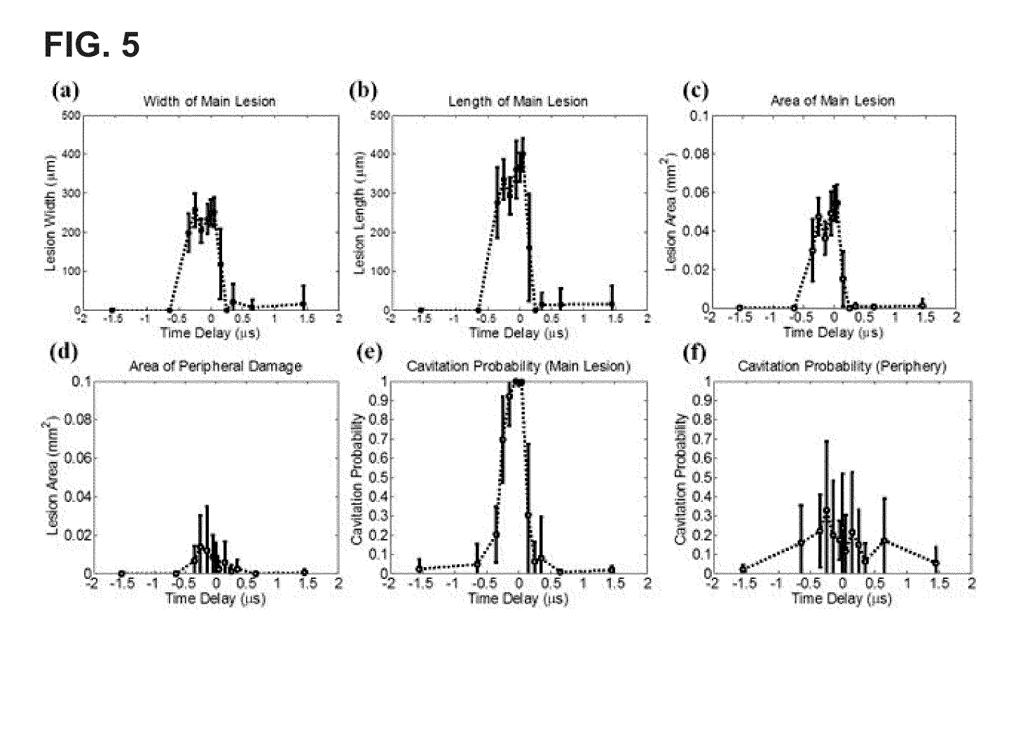

FIGS. 5(a)-(f) show the quantitative result after lesion analysis, including width (FIG. 5(a)), length (FIG. 5(b)), and area (FIG. 5(c)) of the main lesion, area of the peripheral damage (FIG. 5(d)), and cavitation probabilities in main lesion (FIG. 5(e)) and periphery (FIG. 5(f)). As can be seen, the width, length, area, and cavitation probability of the main lesion reached their maxima at 0 μs time delay, i.e., when the low and high-frequency pulses had maximal P− overlap. No significant changes were observed when the time delay changed to −0.05 or 0.05 μs. Note that a negative time delay indicates the P− of the high-frequency (3 MHz) pulse arrives earlier than that of the 500 kHz pulse, and vice versa. When the time delay changed to even more negative or positive, the cavitation probability in the main lesion decreased due to a reduction of combined P−. When the time delays were −0.35, −0.25, −0.15, and 0.15 μs, their cavitation probabilities of the main lesions decreased to between 20% and 95%, and these inconsistent bubble cloud generations led to slightly smaller lesions with higher variability. When the time delay was earlier than −0.50 μs or later than 0.20 μs, the cavitation probability in the main lesion decreased to less than 10% (close to 0% for −1.55 and 1.45 μs time delay), and almost no lesions were observed. The lesion area and cavitation probability for the periphery showed a similar trend. Although their maxima both appeared at −0.25 μs time delay, they did not differ significantly from those at 0 μs.

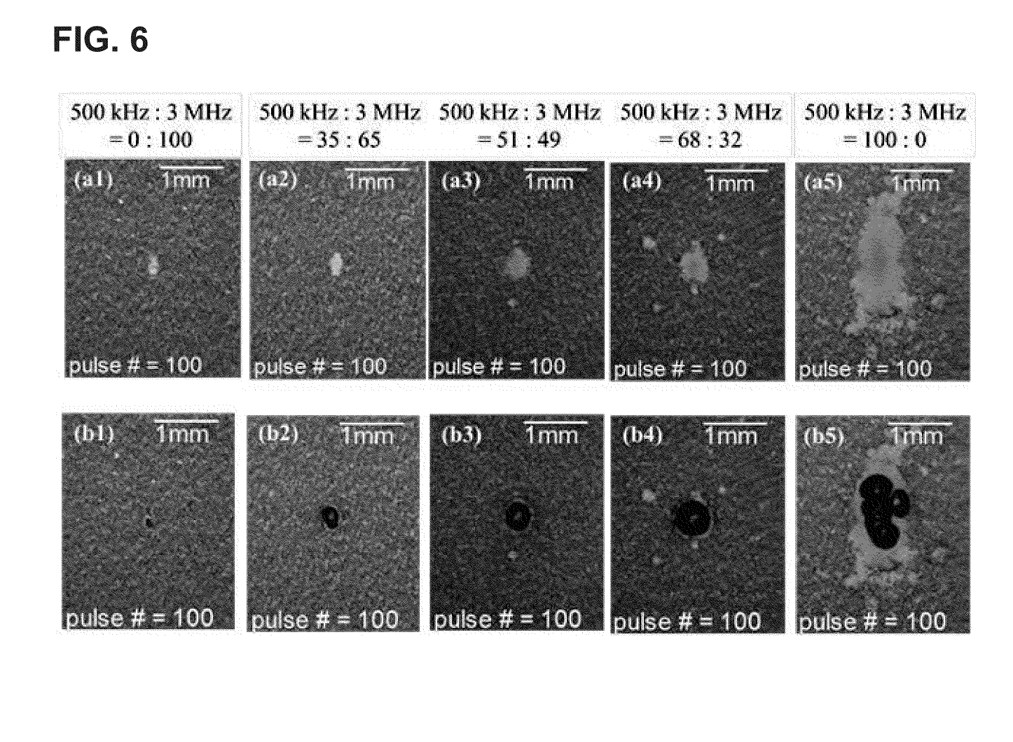

Varying Relative Amplitudes between 500 kHz and 3 MHz Pulses: A total of 5 different combinations, as listed in Table I, were investigated, and each case had a sample size of six. FIG. 6 shows representative lesion and bubble images for each case. As can be seen, the sizes of the lesions and bubble clouds increased as the relative proportion of the 500 kHz pulse amplitude increased (with the relative proportion of the 3 MHz pulse amplitude decreased correspondingly). The labels on the very top of FIG. 6 indicate the corresponding relative amplitude between the low-frequency “pump” and high-frequency “probe” pulses for each column. Figures in the first row (FIGS. 6(a1)-(a5)) show representative lesion images after 100 delivered Histotripsy pulses. Figures in the second row (FIGS. 6(b1)-(b5)) show representative bubble cloud images. All the bubble cloud and lesion images were taken in the axial-lateral plane of the transducer and the Histotripsy pulse propagated from bottom to the top.

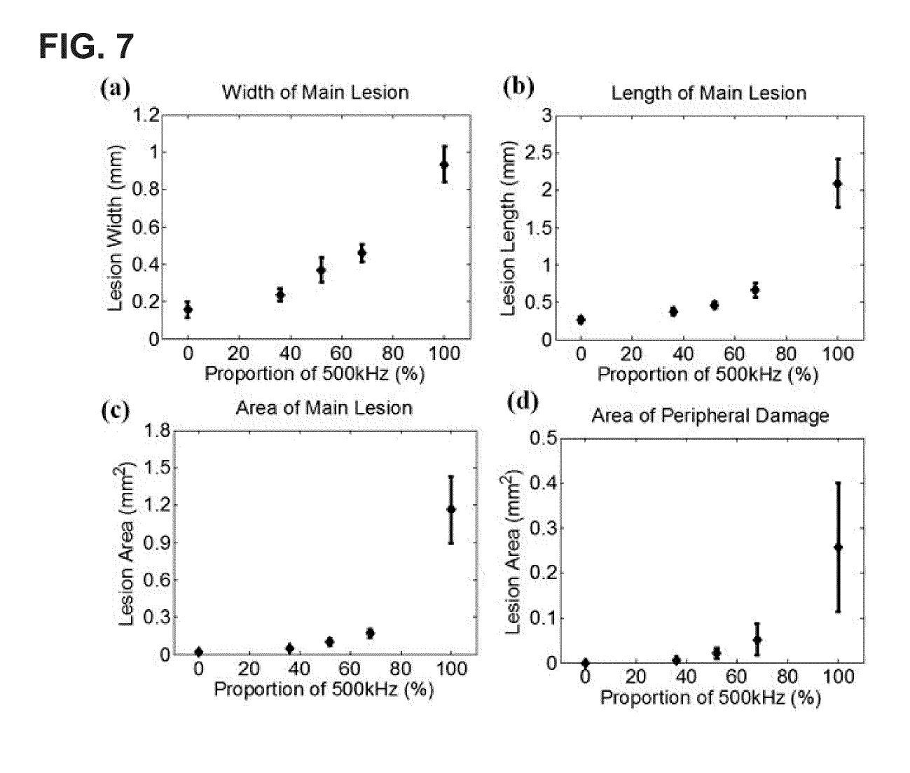

FIGS. 7(a)-(d) summarize the quantitative results for the width (FIG. 7(a)), length (FIG. 7(b)) and area (FIG. 7(c)) of the main lesion and the area of the peripheral damage (FIG. 7(d)). As the relative amplitude proportion of the 500 kHz pulses increased from 0% to 100%, the average lesion sizes increased from 157 μm to 935 μm (the width of the main lesion), 265 μm to 2.094 mm (the length of the main lesion), 0.022 mm2 to 1.163 mm2 (the area of the main lesion), and 0.000 mm2 to 0.257 mm2 (the area of the peripheral damage). The vertical bars in all the Figures represent +/−one standard deviation, and the sample size for each case is six (N=6).

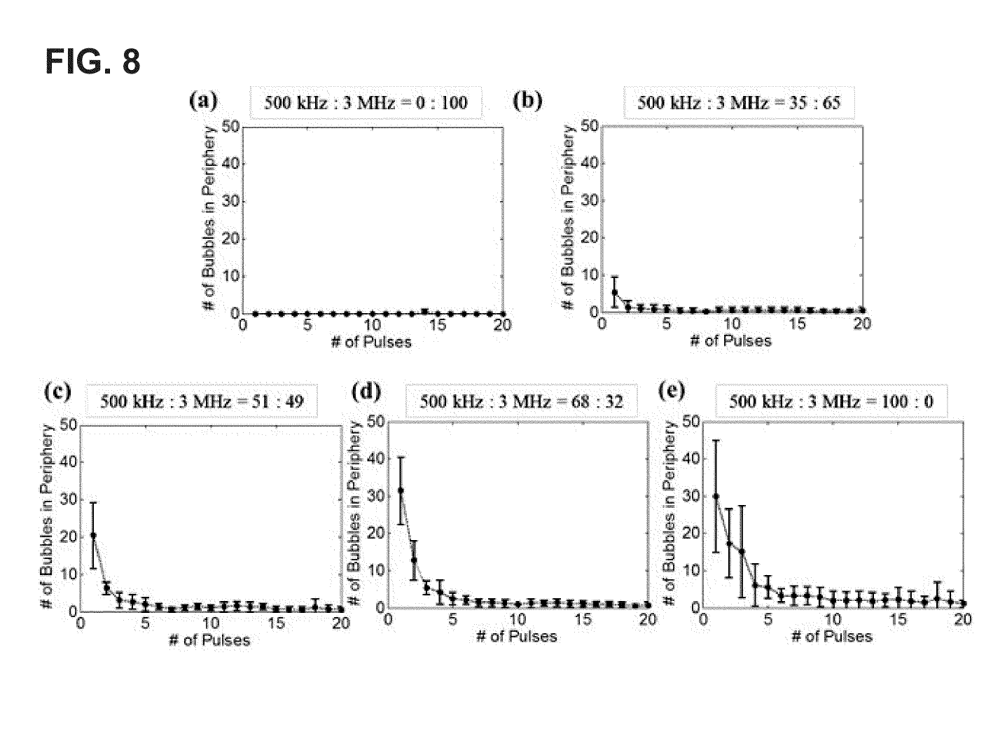

FIGS. 8(a)-(e) show the number of incidental bubbles generated at the periphery, which are responsible for peripheral damage, as a function of the number of applied Histotripsy pulses. As can be seen, the number of these bubbles started from its maximal value and rapidly decreased to almost no bubble presence after the 10th pulse. Additionally, when the 500 kHz pulse amplitude fraction was higher, the number of incidental bubbles at the periphery increased and these bubbles disappeared more slowly. Results after 20 pulses are not plotted since they are similar as the result of the 20^{th} pulse. The vertical bars in all the Figures represent +/−one standard deviation, and the sample size for each case is six (N=6). FIGS. 8(a)-(e) show results from cases in which different relative pulse amplitude proportions were used.

During experimentation, the propagation direction of the low-frequency (500 kHz) and high-frequency (3 MHz) pulses were also varied from co-propagation, orthogonal-propagation, to counter-propagation to investigate effects in lesion production. In this experimental set, all low-frequency transducer elements were firing together with one selected high-frequency element to implement different propagation directions. The arrival times of the low and high-frequency pulses in this experimental set were chosen such that the time delay between the two pulses had maximal P− overlap at the focus of the array transducer (i.e., no time delay or a time delay of 0 μs).

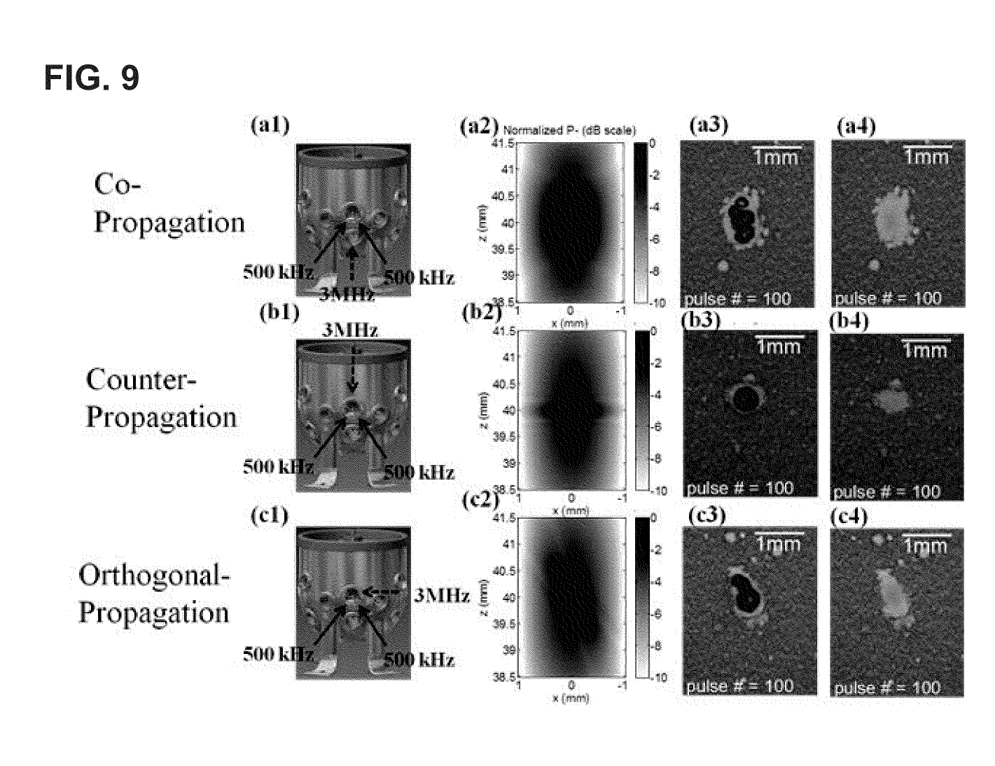

The propagation direction was varied from co-propagation, then counter-propagation, and to orthogonal-propagation, and each had a sample size of eight. FIG. 9 shows the corresponding transducer firing arrangements (FIGS. 9(a1), 9(b1), and 9(c1)), 2D pressure field simulations using FOCUS (FIGS. 9(a2), 9(b2), and 9(c2)), bubble cloud images (FIGS. 9(a3), 9(b3), and 9(c3)), and lesion images (FIGS. 9(a4), 9(b4), and 9(c4)) for different propagation directions. The labels on the very left indicate the corresponding propagation direction for each row. Figures in the first column (9(a1), (b1), and 9(c1)) illustrate the propagation direction of the probe pulse relative to the pump pulse. Figures in the second column (9(a2), (b2), and 9(c2)) show the 2D field patterns (plotting normalized P− in dB scale) using linear transient simulation with “FOCUS”. Figures in the third column (9(a3), (b3), and 9(c3)) show representative bubble cloud images. Figures in the fourth column (9(a4), (b4), and 9(c4)) show representative lesion images after 100 delivered Histotripsy pulses. All the bubble cloud and lesion images were taken in the axial-lateral plane of the transducer and the 500 kHz pulse propagated from bottom to the top. As can be seen, the lesion length in the axial direction for the counter-propagation case was significantly smaller than that for co-propagation case. Also, the lesion shape seemed tilted in the orthogonal-propagation case in comparison to that in the co-propagation case. These results correspond well to the 2D peak negative pressure (P−) field simulation.

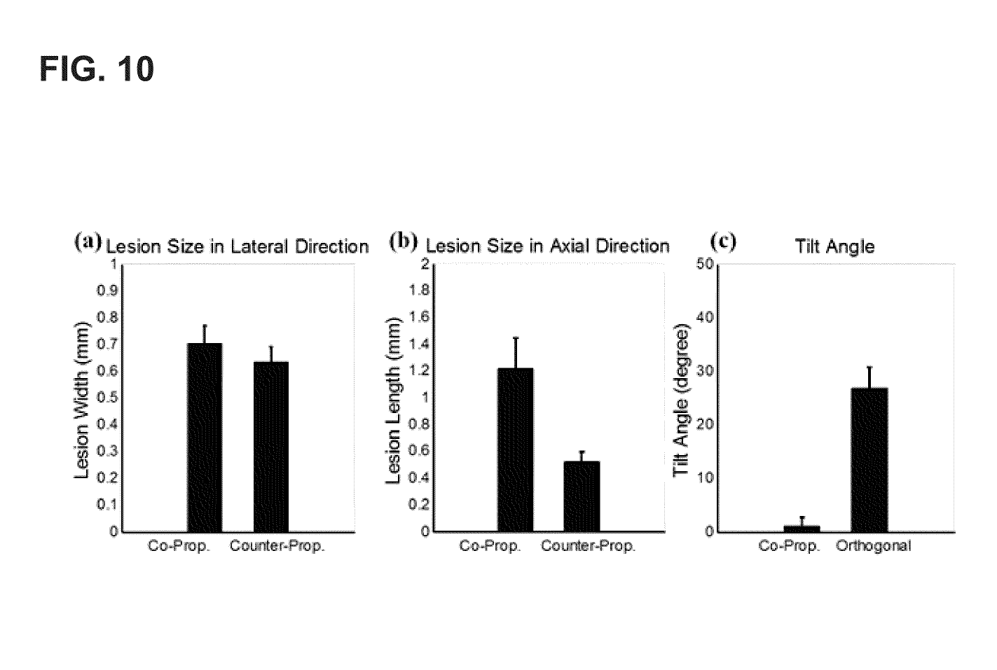

The quantitative analysis of these lesions is summarized in FIGS. 10(a)-(c). The lesion size in the axial direction changed significantly from 1.21 mm for the co-propagation case to 0.52 mm for the counter-propagation case, while the lesion size in the lateral direction remained in similar level (0.70 mm for the co-propagation case and 0.63 mm for the counter-propagation case). The tilt angle changed from 0.99 degrees for the co-propagation case to 26.82 degrees for the orthogonal-propagation case. This tilt angle was quantified by manually selecting the top and bottom points of the lesion, forming a central axis of the lesion, and then calculating the angle between this central axis and the axial propagation direction of the 500 kHz pulse component. The vertical bars in all the Figures represent +one standard deviation, and the sample size for each case is eight (N=8). FIG. 10(a) shows a comparison between co-propagation and counter-propagation for the width (lesion size in the lateral direction) of the main lesion. FIG. 10(b) shows a comparison between co-propagation and counter-propagation for the length (lesion size in the axial direction) of the main lesion. FIG. 10(c) is a comparison between co-propagation and orthogonal propagation for the tilt angle of the main lesion.

The relative amplitudes of the low-frequency (500 kHz) and high-frequency (3 MHz) pulses were varied to study the smallest reproducible lesions for each combination. The arrival times of the 500 kHz and 3 MHz in this experimental set were chosen to be the time delay when the two produced the maximal P− values of the composite waveforms (i.e., 0 μs as established by the convention specified above).

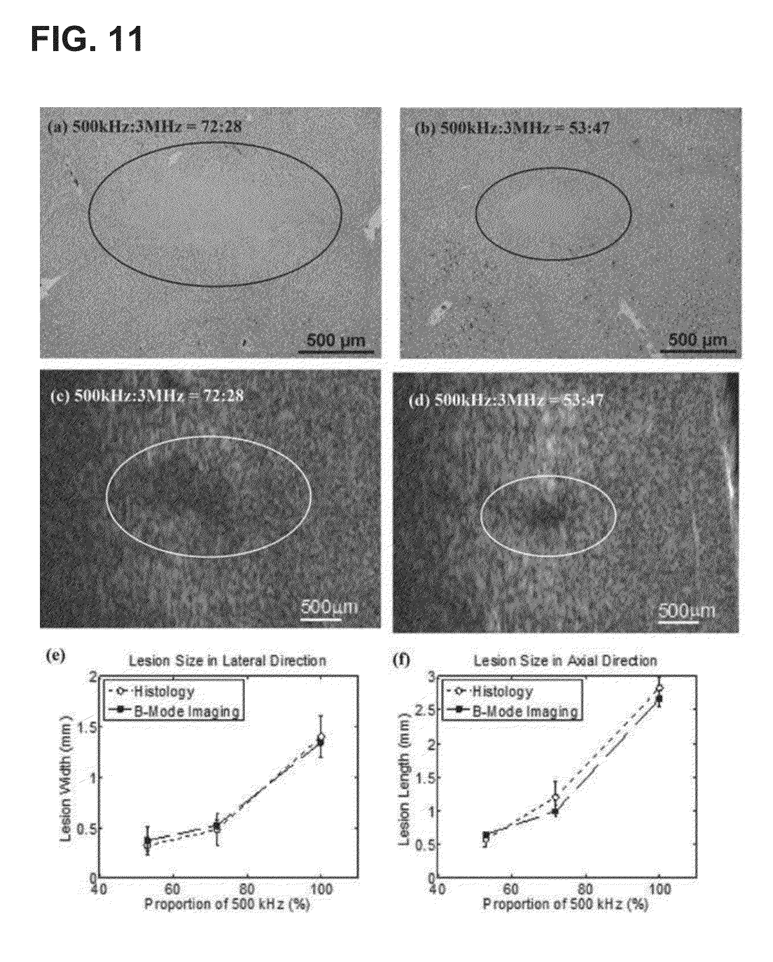

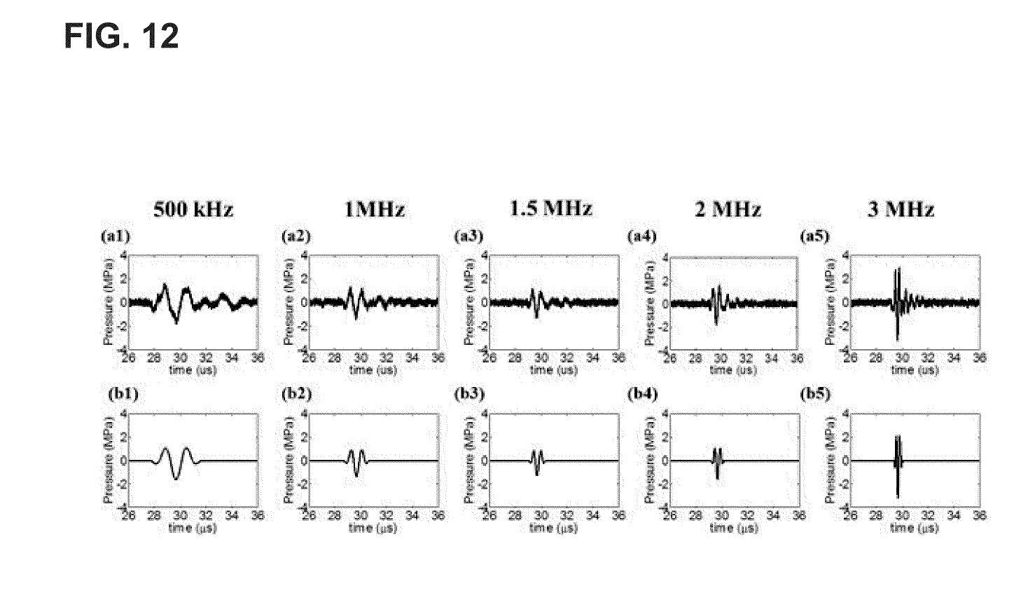

A total of 3 different pressure combinations, as listed in Table II above, were used to generate lesions in ex vivo porcine hepatic specimens, and each case had a sample size of two (N=2). The representative histological sections are displayed in FIGS. 11(a) and 11(b). The intended treatment regions had lost their normal architecture and contained only acellular granular debris, and a larger spatial extent of the lesion occurred when a higher proportion of the 500 kHz pulse was applied. FIGS. 11(c) and 11(d) show representative B-mode images of the hepatic specimens after the application of 500 Histotripsy pulses. Note that the B-mode ultrasound images were rotated 90 degrees from their original orientations in order to match the orientations of the histological sections. Hypoechoic regions occurred on B-mode images after Histotripsy treatment, and these regions were larger when a higher proportion of 500 kHz pulse was applied. The histological sections and B-mode images for the case with 100% proportion of 500 kHz pulse are not displayed in FIG. 11 since this disclosure focuses on using both low-frequency pump (500 kHz) and high-frequency probe (3 MHz) pulses. The quantified lesion sizes in the lateral and axial directions are shown in FIGS. 12(e) and 12(f), respectively. As can be seen, the quantified lesion sizes increased as the proportion of the 500 kHz pulse increased and the results quantified from histological sections and B-mode images were close to each other.

The representative histological section and B-mode image for 500 kHz:3 MHz=72:28 are shown in FIGS. 11(a) and (c), respectively. The representative histological section and B-mode image for 500 kHz:3 MHz=53:47 are shown in FIGS. 11 (b) and (d), respectively. Histological sections and B-mode ultrasound images were both taken in the axial-lateral plane of the transducer and Histotripsy pulses propagated from right to the left. The B-mode ultrasound images were rotated 90 degrees from their original orientations in order to match the displayed orientations of the histological sections. The quantified lesion sizes in the lateral and axial directions are shown in (e) and (f), respectively. Empty circles together with dotted lines (...∘...) indicate results quantified from histological sections, while solid squares together with dashed lines (---▪---) indicate results quantified from B-mode images. The vertical bars in these two Figures represen t+/−one standard deviation, and the sample size for each case is two (N=2).

In this disclosure, precise lesions were generated by “parametric Histotripsy” pulses using the intrinsic threshold mechanism tissues. The parametric Histotripsy pulse is comprised of a low-frequency “pump” pulse and a high-frequency “probe” pulse wherein a proper time delay between the two is chosen to allow their P− values to add constructively at the focus so as to exceed the intrinsic cavitation threshold in tissue. As can be seen in FIGS. 4 and 5, when the pump and probe pulses had maximal P− overlap (i.e., 0 μs time delay), consistent bubble clouds were generated with a cavitation probability of 100% and the size of the main lesion reached its maximum. No significant changes in the cavitation probability and lesion size were observed when the probe pulse (3 MHz) arrived 0.05 μs earlier or later than the pump pulse (500 kHz). When the time delay between the P−'s of the pump and probe pulses increased to 0.15 μs or more, the diminution of the combined P− led to decreases in the cavitation probability and lesion size (with higher variability), and both the cavitation probability and lesion size approached 0 when the negative phases of the pump and probe pulses did not have any overlap. Furthermore, these decreases were not symmetric around 0 μs time delay, which was likely due to the negative pressure phase for the 500 kHz pulse (in time domain) not being symmetric, as can be seen in FIG. 2(a). This asymmetry in the 500 kHz waveform was probably not a result of nonlinear propagation since it also occurred at really low applied pressure level. Imperfection in the fabrication stacking process is a potential explanation since it only occurred in the epoxy-stacked 500 kHz elements.

Additionally, the size of the smallest reproducible lesions decreased when a higher proportion of the high-frequency “probe” pulse (3 MHz) was applied, as indicated in FIGS. 6 and 7. With only 32% of the probe pulse, the lesion size decreased significantly from where pulses with 100% 500 kHz were applied. The lesion width decreased from 0.93 to 0.46 mm, the lesion length decreased from 2.09 to 0.66 mm, and the lesion area decreased from 1.16 to 0.17 mm2. This demonstrates that, with the addition of a minor portion of the probe pulse, significantly smaller lesions can be achieved, in comparison to 100% pump pulse.

Moreover, the size and shape of the produced lesions can be further manipulated using various propagation directions between pump and probe pulses. (1) The axial dimension of the lesion can be further reduced when a “probe” pulse counter-propagates with a “pump” pulse, as shown in FIGS. 9 and 10. This “foreshortening” of the lesion results from the very short interaction time window when two short acoustic pulses (only one large negative pressure phase in the 2-cycle pulses) counter-propagate with each other (note that CW waves would not produce the same effect). (2) When a probe pulse orthogonally-propagates with a pump pulse, the lesion can be “tilted” from the propagation axis of the pump pulse. As shown in FIGS. 9(c1)-9(c4), when the pump pulse propagates from the bottom to the top and the probe pulse propagates from the right to the left, they firstly interact in the lower right corner of the focus. As they propagate through the focus, the two pulses produce a supra-threshold P− value moving from the lower right to the upper left, making the lesion appear tilted from the propagation axis of the pump pulse.

Though using propagation directions other than co-propagation might not work in many applications due to the lack of accessible acoustic windows, it might still be applicable in some situations. For example, in one embodiment a dual-frequency ultrasound therapy transducer can be used for prostatic tissue ablation. A transrectal low-frequency “probe” pulse can be counter-propagated with a transabdominal “pump” pulse to ablate prostatic tissue. Counter- or orthogonal-propagation of catheter-based probe pulses with transcostal/transabdominal pump pulses an also be used in cardiac or hepatic tissue treatment allowing pulses from small-aperture high-frequency transducers to reach threshold levels not possible when used alone.

Peripheral damage induced by the incidental bubbles generated at the periphery of the focus was observed in both single-frequency Histotripsy and parametric Histotripsy (“pump” plus “probe” pulses). These incidental bubbles almost disappeared by the time the 20th pulse was applied. A higher proportion of the pump pulse led to a larger area of peripheral damage, a larger number of incidental bubbles, and a slower rate in the decrease of the number of incidental bubbles. These incidental bubbles were likely seeded from the pre-existing dissolved sub-micron gas bubbles (samples could not be 100% degassed) or weak pockets. The application of Histotripsy pulses firstly excited these weak nuclei at the periphery (where P− was below the intrinsic threshold) and then subsequently destabilized them, causing the incidental bubbles to disappear quickly. After that, the bubble clouds were preferentially generated at the location where the main lesion was forming. This “self-quenching” phenomenon limits the damage in the periphery, containing the lesion primarily to the volume where P− exceeds the intrinsic threshold.

Thus, “parametric Histotripsy” can be quite beneficial in situations where precise treatment is required through a highly aberrative and attenuative medium. In the situation covered in this disclosure, the pump pulse is highly focused and can only cover a small region (−6 dB beamwidths: 4.9×1.7×1.8 mm) or “target” for the probe pulse. An imaging probe parametric Histotripsy system could use available small windows, e.g., the transcostal region between ribs, to generate precise high frequency steerable pulses “enabled” by a much larger low-frequency pump transducer (e.g., covering much of the rib cage). The use of a high frequency imaging transducer to generate precise lesions has many other interesting applications.

In this study, the capability of “parametric Histotripsy” pump and probe pulses for precise lesion formation is demonstrated both in RBC phantoms and ex vivo porcine tissues. Parametric Histotripsy is accomplished by the application of a low-frequency pump pulse that enables a high-frequency probe pulse to exceed the intrinsic cavitation threshold. With an adjustment in arrival times that allows constructive P− addition at the focus, sub-intrinsic threshold pump and probe pulses can induce dense bubble cloud generation when P− summation exceeds the intrinsic cavitation threshold. The size of the smallest reproducible lesions decreases when the proportion of the probe pulse increases. Counter-propagation of the pump and probe pulse could foreshorten the lesion size in the axial direction. Parametric Histotripsy can be useful in clinical applications where precise tissue ablation is required with a longer propagation depth or through a highly attenuative or aberrative medium, such as transcranial therapy. With small low-attenuation “windows,” even imaging transducers providing the probe pulses could generate Histotripsy lesions.

In the disclosure below, novel techniques are described which synthesize extremely short, “monopolar” (half-cycle) pulses using an ultrasound transducer comprising an array of broadband elements each operating at its own resonant frequency. The resultant acoustic pulse can be synthesized by “frequency compounding,” or combining the output of all the elements constructively in the target volume or tissue. Each individual element of the transducer array can be configured to generate short acoustic pulses (e.g., 1.5 cycles), at its own frequency, using a high voltage pulse generator. By adjusting time delays of individual frequency components to allow their principal peak negative pressures (P−) to align temporally, high peak negative pulses can be generated by constructive addition, or compounding. Destructive interference can occur outside the peak-negative-overlapped temporal window, resulting in a good approximation of a monopolar (half-cycle) pulse with a “sharp” high-amplitude negative phase and low-amplitude “smeared out” positive phases preceding and following the negative phase (a “negative-polarity” pulse). A similar monopolar pulse with a sharp dominant positive phase (a “positive-polarity” pulse) could be generated in a similar way by constructive compounding of all principal peak positive pressures (P+) from all the elements.

The term, “frequency compounding,” has been used for a technique in diagnostic ultrasound wherein speckle noise is reduced by averaging multiple images created from signals filtered at different center frequencies and bandwidths. Acoustical speckle, which appears as a random mottled granular pattern, arises from the coherent interference of multiple echoes from small scatterers within a resolution volume of the imaging system. This speckle noise reduces the perceived resolution and degrades minimum detectable contrast level; moreover, it is time-independent so that the speckle noise cannot be reduced by temporal averaging. Two major approaches, spatial compounding and frequency compounding, have been investigated to “smear out” the speckle effect, allowing the speckle noise to be averaged and minimized. Spatial compounding translates an imaging transducer and creates images from several angular views, while frequency compounding uses images created at different center frequencies and bandwidths as discussed earlier. Although the frequency compounding technique in this disclosure shares the same name as the one for speckle noise reduction, their implementations are fundamentally different. Instead of averaging images created at various frequency bands, this technique is implemented at the level of pulse generation wherein short acoustic pulses with various frequencies are concurrently launched and temporally aligned to approximate monopolar pulses.

Additionally, the frequency compounding technique of this disclosure is different from prior work wherein ultrasound waves with 2^{nd} harmonic superimposition onto the fundamental frequency was applied to enhance cavitation effects. The ultrasound waves used in these previous studies were either continuous waves or long pulses, and only two frequencies were used in the compounding process. In contrast, the technique proposed in this disclosure utilizes short acoustic pulses with a wide variety of frequencies to approximate extremely short monopolar pulses.

The monopolar pulses of this disclosure can be applicable in both therapeutic and diagnostic ultrasound. For Histotripsy therapy, using monopolar pulses with a dominant negative phase can eliminate the shock-scattering effect because no high peak positive shock fronts develop. As a result, the generation of a dense bubble cloud can solely depend on the applied negative half cycle exceeding the intrinsic threshold in the target or tissue, making produced lesions even more controllable, predictable, and small by using this approach.

In diagnostic ultrasound, these monopolar waveforms can be used as the transmit pulses to enhance axial resolution of the imaging due to the reduced pulse length (half cycle). These monopolar pulses would also decrease and minimize speckle noise since these pulses have minimal oscillatory components, leading to less coherent constructive/destructive interference patterns in the image.

These monopolar pulses can also be useful in pulse inversion contrast imaging. In pulse inversion imaging, a sequence of two ultrasound pulses can be transmitted with a proper time delay between the two, the second pulse being an inverted copy of the first one. For a linear medium, the response to the second pulse can be an inverted copy of the response to the first one, and the summation of the two responses can become zero. For a nonlinear target, the response to the second pulse may not be the exact opposite of the response to the first pulse, leading to a non-zero summation. The produced pulse inversion image can then be a map of the nonlinearity of the imaged medium. These monopolar pulses should amplify the difference between object responses when it is exposed to a negative-polarity pulse (mostly rarefactional) and a positive-polarity pulse (mostly compressional), thus increasing the sensitivity of the pulse inversion imaging.

In this disclosure, a multi-element ultrasound transducer, such as the system and transducer in FIG. 1, is described that can be configured to insonify tissue with various resonant frequencies in a frequency compounding technique for monopolar pulse generation.

In one embodiment, a transducer array having a plurality of piezoceramic elements configured to transmit ultrasound pulses at various resonant frequencies, ranging from 100 kHz up to 10 MHz (e.g., 0.5, 1, 1.5, 2, and 3 MHz) to implement the frequency compounding technique of this disclosure for monopolar pulse generation. One or more transducer elements can be configured to transmit ultrasound energy at various resonant frequencies. For example, one or more transducer arrays can have various resonant frequencies, e.g., 500 kHz, 1 MHz, 1.5 MHz, 2 MHz, and 3 MHz.

The generation of monopolar pulses with a dominant negative phase (negative-polarity pulses) was investigated by adjusting the arrival times of individual frequency components to allow their principal negative phase peaks to arrive at the focus of the transducer concurrently. In this situation, destructive interference occurs elsewhere in space and time, leading to a diminution of the peak positive pressure of the combined ultrasound pulse. For the generation of monopolar pulses with a dominant positive phase (positive-polarity pulses), driver pulses for the individual elements were inverted, resulting in ultrasound pulses with a single principal positive phase from each element. The arrival times of individual frequency components were then adjusted to allow their principal positive phase peaks to arrive at the focus concurrently.

Two experimental sets were performed, one was the exposure to negative-polarity pulses and the other was the exposure to positive-polarity pulses. Each intended treatment region was exposed with 200 pulses at a PRF of 1 Hz, and a single-focal-point exposure was performed. Applied pressure levels are listed in Table III.

TABLE III
APPLIED PRESSURE LEVELS AND TIME DELAYS FOR CAPTURING
BUBBLE CLOUD IMAGES USED IN RBC PHANTOM EXPERIMENTS

| | | | | | | | Focal P− of Linearly | Time Delay For |
|---|---|---|---|---|---|---|---|---|
| | | | | | | | Summed Signal with | Capturing |
| Experimental | | Individual Focal P− with Attenuation Correction* (MPa) | | | | | Attenuation | Bubble Cloud |
| Set | Case | 500 kHz | 1 MHz | 1.5 MHz | 2 MHz | 3 MHz | Correction* (MPa) | Images (μs) |
| 1 | 1 | 7.4 | 5.4 | 4.9 | 3.8 | 8.8 | 27.6 | 4 |
| | 2 | 8.9 | 6.7 | 6.1 | 4.6 | 10.4 | 33.2 | 6 |
| | 3 | 10.2 | 8.0 | 7.2 | 5.2 | 11.9 | 39.0 | 9 |
| | 4 | 12.0 | 9.4 | 8.2 | 5.8 | 13.4 | 44.3 | 12 |
| | 5 | 13.7 | 10.5 | 9.1 | 6.2 | 14.7 | 48.9 | 17 |
| | | | | | | | Focal P+ of Linearly | Time Delay For |
| | | | | | | | Summed Signal with | Capturing |
| Experimental | | Individual Focal P+ with Attenuation Correction* (MPa) | | | | | Attenuation | Bubble Cloud |
| Set | Case | 500 kHz | 1 MHz | 1.5 MHz | 2 MHz | 3 MHz | Correction* (MPa) | Images (μs) |
| 2 | 1 | 7.5 | 5.2 | 5.9 | 5.5 | 11.9 | 30.4 | 4 |
| | 2 | 9.9 | 7.1 | 8.4 | 7.1 | 15.0 | 36.8 | 6 |
| | 3 | 11.8 | 9.1 | 11.3 | 8.4 | 17.5 | 42.3 | 9 |
| | 4 | 14.0 | 11.5 | 14.0 | 9.8 | 21.2 | 48.0 | 12 |

*The pressure levels were corrected by the attenuation contributed by the plastic gel holders and agarose hydrogels.
The attenuation contributed by the plastic gel holders was measured using the FOPH.
The attenuation contributed by agarose hydrogels was calculated using a reported attenuation coefficient and assuming linear propagation.

Representative individual temporal focal waveforms in free-field for each frequency component are plotted in FIG. 12 (the results from the FOPH measurement and the FOCUS simulation are both plotted). As can be seen from the FOPH-measured waveforms (FIGS. 12(a1)-(a5)), the FPGA-controlled high voltage pulser enabled individual frequency components to output short acoustic pulses with only one principal negative phase. Note that the actual waveforms were slightly longer than the simulated waveforms.

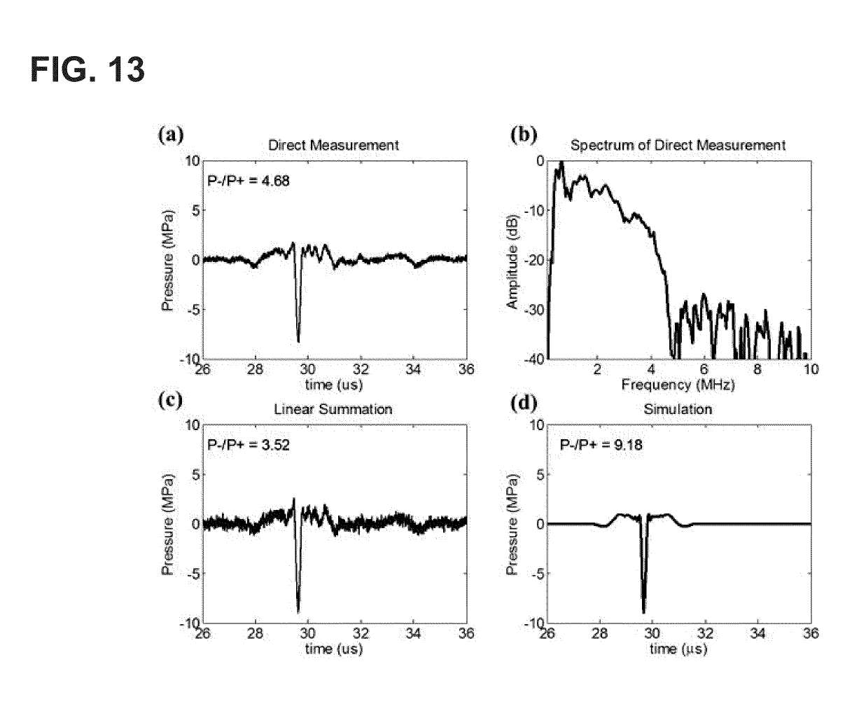

The generation of negative-polarity pulses was investigated by adjusting the arrival times of individual frequency components to allow their peaks of principal negative phases to arrive at the focus concurrently. FIGS. 13(a)-(d) plot representative waveforms in free-field of a negative-polarity pulse, including a waveform directly measured using the FOPH with all frequency components firing simultaneously (FIG. 13(a)), a frequency spectrum of the directly-measured waveform (FIG. 13(b)), a linearly summed waveform using FOPH-measured waveforms of individual frequency components (FIG. 13(c)), and a simulated waveform using the FOCUS simulation tool (FIG. 13(d)). These pulses were generated using the same amplitudes for individual frequency components as those in FIG. 12. As can be seen in FIG. 13, an approximately monopolar pulse with a dominant negative phase was generated, and the ratio of P− to P+ was 4.68 for the directly measured waveform (3.52 for the linearly summed waveform and 9.16 for the simulated waveform). The temporal full-width-half-maximum (FWHM) of the negative phase of the directly measured waveform was 0.17 μs, which was in between FWHMs of the principal negative phases of the 2 MHz (0.19 μs) and 3 MHz (0.11 μs) components.

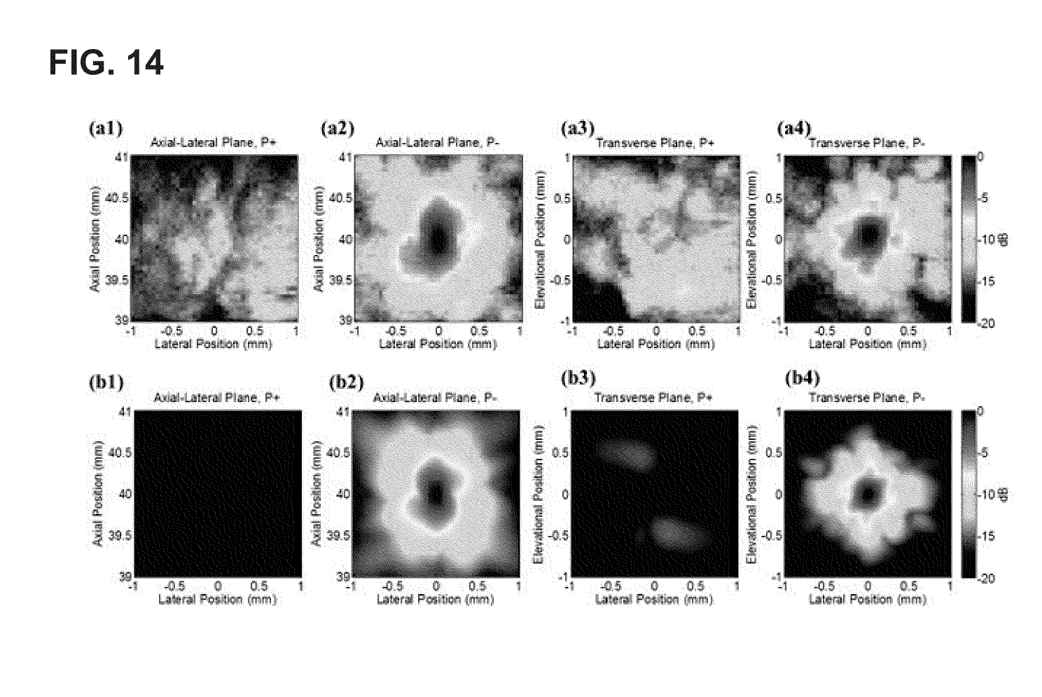

FIG. 14 shows representative 2D spatial pressure fields for a negative-polarity pulse, including pressure fields directly measured using the FOPH with all frequency components firing simultaneously (FIGS. 14(a1)-(a4)) and pressure fields simulated using the FOCUS simulation tool (FIGS. 14(b1)-(b4)). In this 2D pressure field measurement, the negative-polarity pulse had a P− of 18.1 MPa at the focus, and the 2D pressure fields for P− and P+ were both normalized to the absolute value of the P− at the focus. The relative amplitudes of individual frequency components were the same as the ones used in FIGS. 12 and 13. As can be seen in FIG. 14, the FOPH-measured 2D P− pressure fields show a good agreement with the FOCUS-simulated 2D P− pressure fields, in both the axial-lateral and transverse planes. The 2D P+ pressure fields had a slight variation between FOPH-measured ones and FOCUS-simulated ones, possibly due to the limitation in sensitivity of the FOPH (predominantly noise signal at low pressure level) or the actual pulses not being ideally short as the pulses used in the simulation (as seen in FIG. 12).

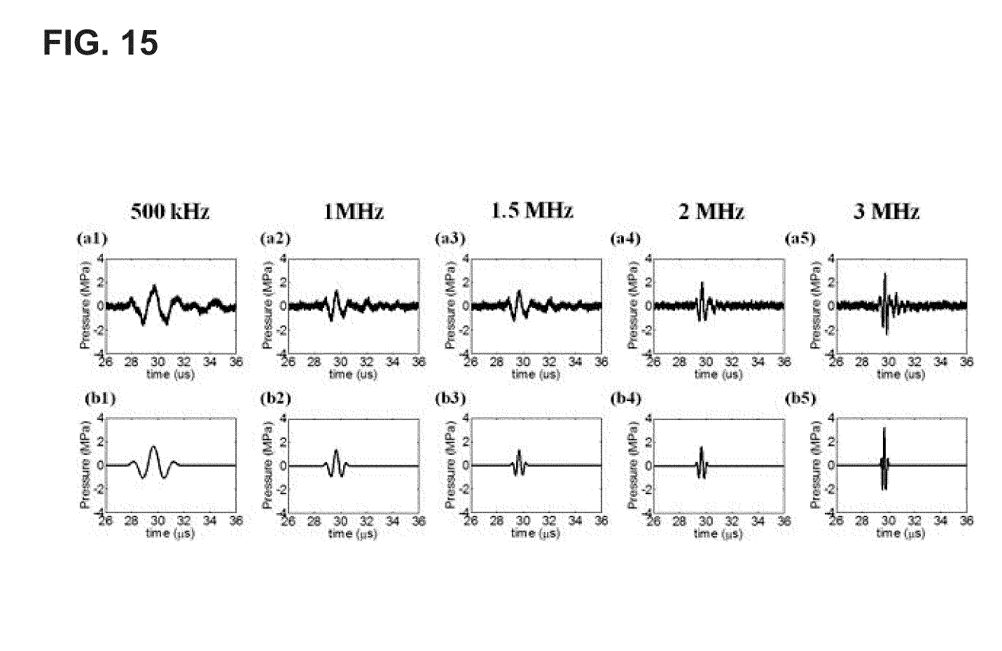

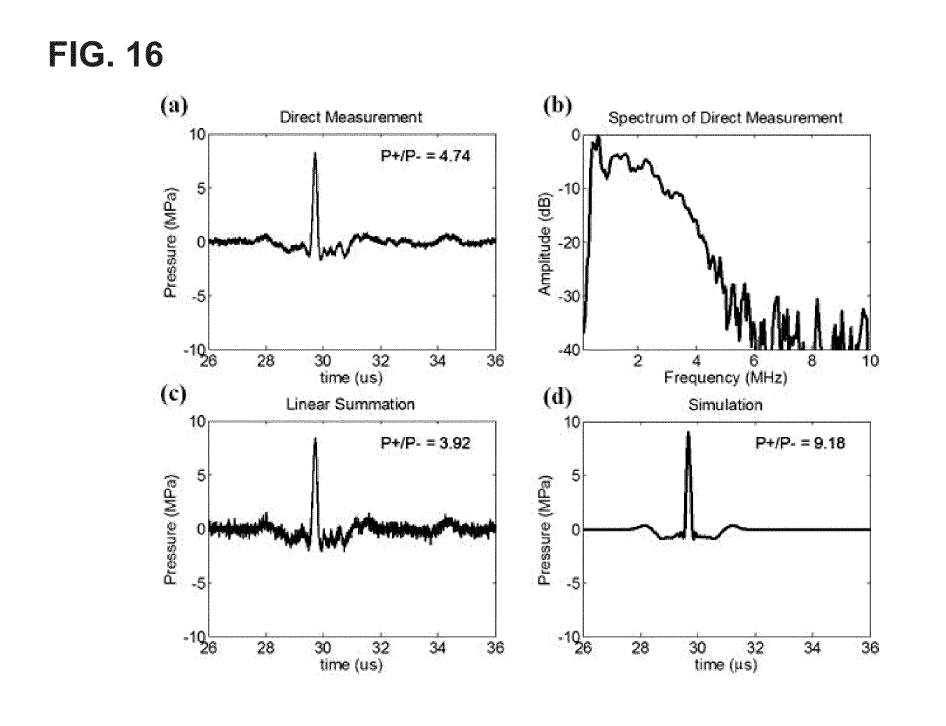

In one embodiment, all driving signals are inverted, resulting in an inversion of the output focal waveforms. FIG. 15 plots representative FOPH-measured and simulated temporal focal waveforms for individual frequency components. As can be seen, inverting the output of the FPGA-controlled high voltage pulse generator enabled each frequency component to output short acoustic pulses with only one principal positive phase. The arrival times of each frequency component can be adjusted to allow their positive phase peaks to arrive at the focus simultaneously. This resulted in a nearly monopolar pulse with a dominant positive phase (a positive-polarity pulse), shown in FIG. 16, and in one embodiment the ratio of P+ to P− was 4.74 for the directly measured waveform.

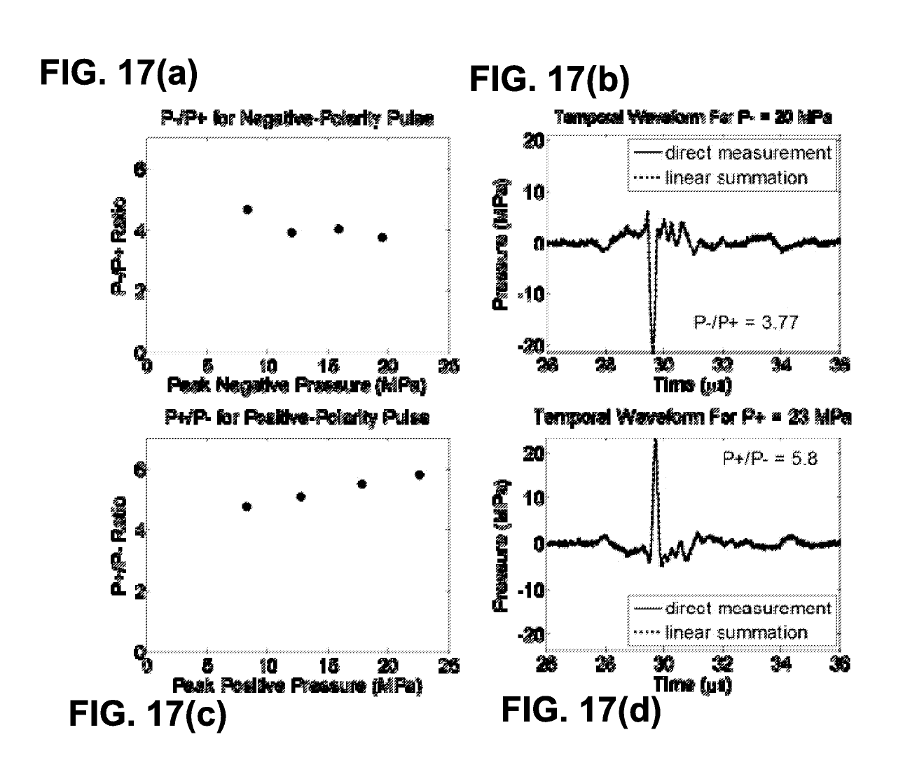

FIGS. 17(a) and 17(b) plot the ratio of P− to P+ as a function of applied pressure level (P−) for negative-polarity pulses and a representative temporal focal waveform at P−=20 MPa, respectively. FIGS. 17(c) and 17(d) plot the ratio of P+ to P− as a function of applied pressure level (P+) for positive-polarity pulses and a representative temporal focal waveform at P+=23 MPa, respectively. As can be seen in FIGS. 17(a) and 17(c), when the pressure levels increased, the ratio of P− to P+ for the negative-polarity pulses decreased slightly, while the ratio of P+ to P− for the positive-polarity pulses increased slightly. This can be due to an increase in nonlinear propagation effects at higher pressure levels. In FIGS. 17(b) and 17(d), the directly measured waveforms had a good agreement with the linearly summed waveforms at a higher pressure level for both negative-polarity and positive-polarity pulse cases, and the shapes of these directly measured waveforms did not significantly deviate from the ones measured at a lower pressure level (e.g., FIGS. 13(a) and 16(a)).

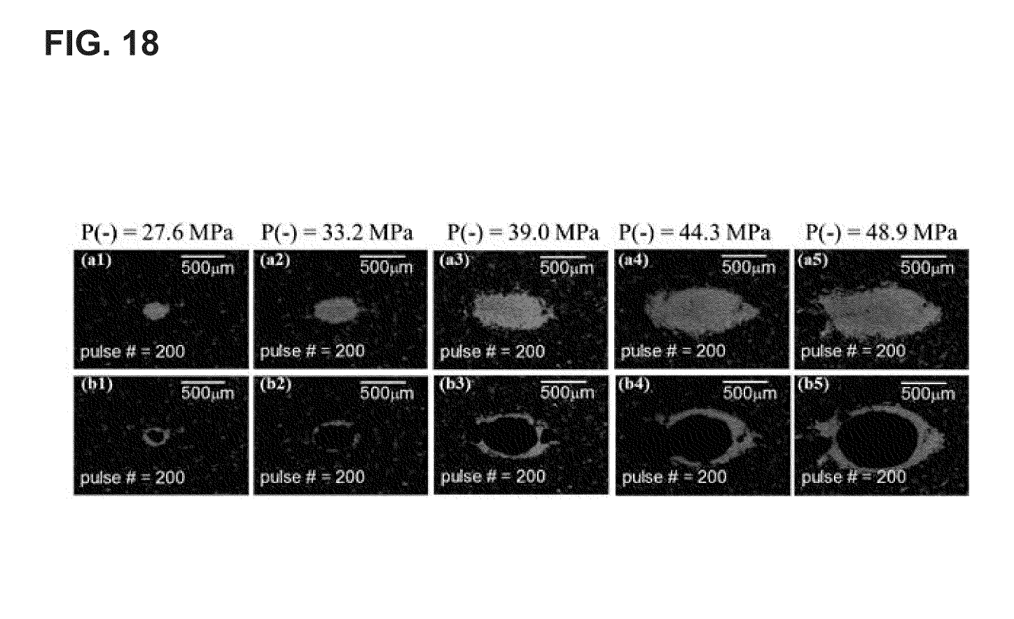

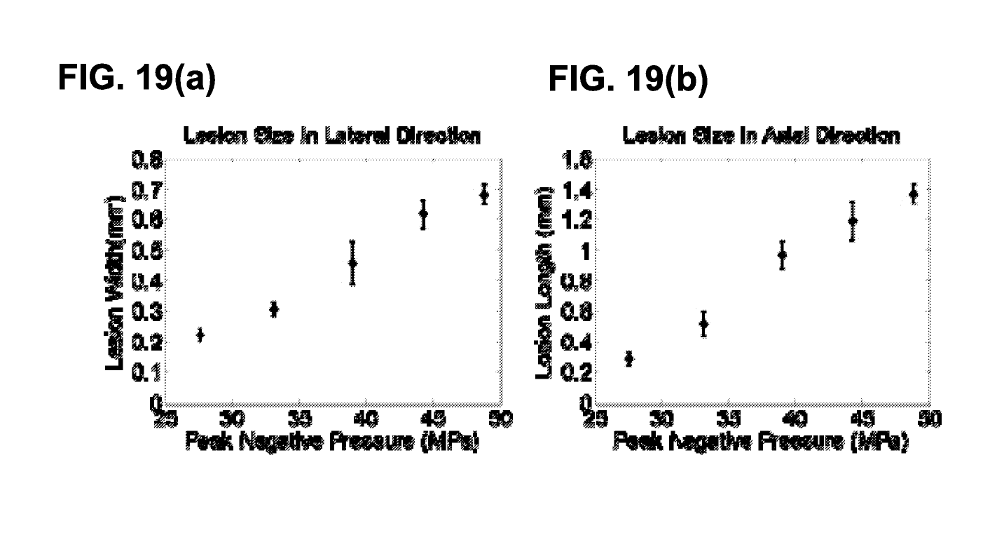

During experimentation, a total of 30 lesions were generated using negative-polarity pulses (six for each pressure level listed in Table III), and their representative lesion and bubble cloud images are shown in FIG. 18. Their quantified lesion sizes in the lateral and axial directions are plotted in FIGS. 19(a) and 19(b), respectively. As can be seen from FIGS. 18 and 19, cavitation-induced lesions were observed using negative-polarity pulses with P− exceeding the intrinsic threshold, and the sizes of the generated lesions and the bubble clouds increased as the applied P− increased.

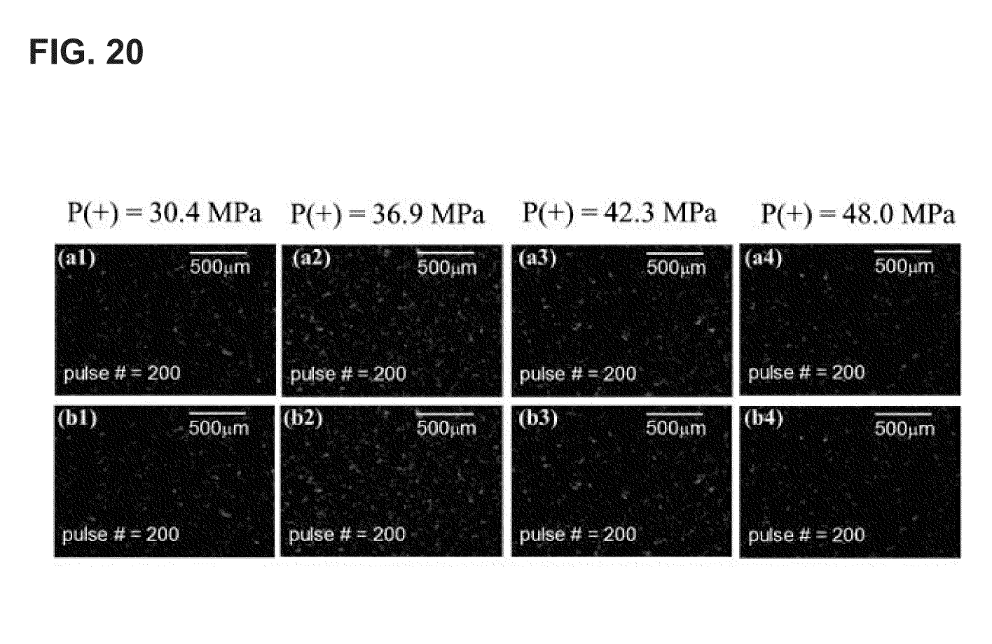

A total of 20 locations were exposed to positive-polarity pulses (five for each pressure level listed in Table III), and their representative lesion and bubble cloud images are shown in FIG. 20. As can be seen, neither lesions nor bubble clouds were generated using positive-polarity pulses with P+ ranging from 30 to 50 MPa. No further quantitative lesion analysis was performed for the positive-polarity pulse case.

In this disclosure, the synthesis of approximately “monopolar” pulses was demonstrated using a frequency-compounding transducer having a plurality of transducer elements that transmit at different resonant frequencies, e.g., 0.5, 1, 1.5, 2, and 3 MHz. By properly adjusting time delays for individual frequency components, monopolar pulses could be generated. The temporal FWHM of the generated negative-polarity pulse was between the temporal FWHMs of the negative phases of the 2 MHz and 3 MHz components. This shows that the frequency of the combined monopolar pulses lies within the frequency span of the comprising frequency components. The frequency components (500 kHz to 3 MHz) in a frequency-compounding transducer were selected since these frequency components are ones commonly used in Histotripsy therapy, although this selection could be optimized for different applications. For example, for imaging or therapy at deeper targets with highly attenuative and aberrative intervening tissue, lower frequency components could be chosen to generate lower-frequency monopolar pulses. On the other hand, for superficial and microscopic targets, higher frequency components could be applied to generate higher-frequency monopolar pluses.

Moreover, the generation of monopolar pulses can be limited to the focus of the frequency-compounding transducer. The design of a highly focused transducer (an f-number of close to 0.5, hemispherical) can be chosen to allow the transducer to provide sufficient output pressure for Histotripsy therapy (this current transducer is capable of generating a combined P− of 100 MPa). In diagnostic ultrasound, this level of output pressure would be prohibited due to safety concerns; therefore, a different approach could be adopted when designing a frequency-compounding transducer for imaging purposes. This design could favor a higher f-number configuration in order to cover an imaging plane more uniformly. For some applications, an ideal array transducer would emit a plane wave consisting of a frequency-compounded monopolar pulse. This could be a linear array with subsets of modules wherein each module alone can generate monopolar pulses with a desired frequency. Some recent advances in transducer array manufacturing involving micro fabrication of piezoceramic components using microstereolithography could benefit the development of frequency-compounding transducers comprising microelements with various resonant frequencies.

As shown in FIG. 17, when the combined pressure level increased from ˜8 MPa to −23 MPa, the ratio of P− to P+ for negative-polarity pulses slightly decreased and the ratio of P+ to P− for positive-polarity pulses slightly increased. This slight change in P− and P+ ratios is likely due to nonlinear propagation. However, this nonlinear effect was minimal at the highest pressure level shown in FIG. 17, since the changes in the ratios of P− and P+ values were minimal and the shapes of the focal pressure waveforms did not deviate significantly from those at the lower pressure level shown in FIGS. 13(a) and 14(a). Separating elements with the same resonant frequency in the current transducer arrangement likely contributes to this minimal development of nonlinearity.

Some high f-number transducers are limited in peak amplitude by “nonlinear saturation” wherein higher order harmonics are attenuated rapidly as driving signals increase. Frequency-compounding arrays result in minimal constructive interference outside the focal zone, thus reducing nonlinear saturation effects.

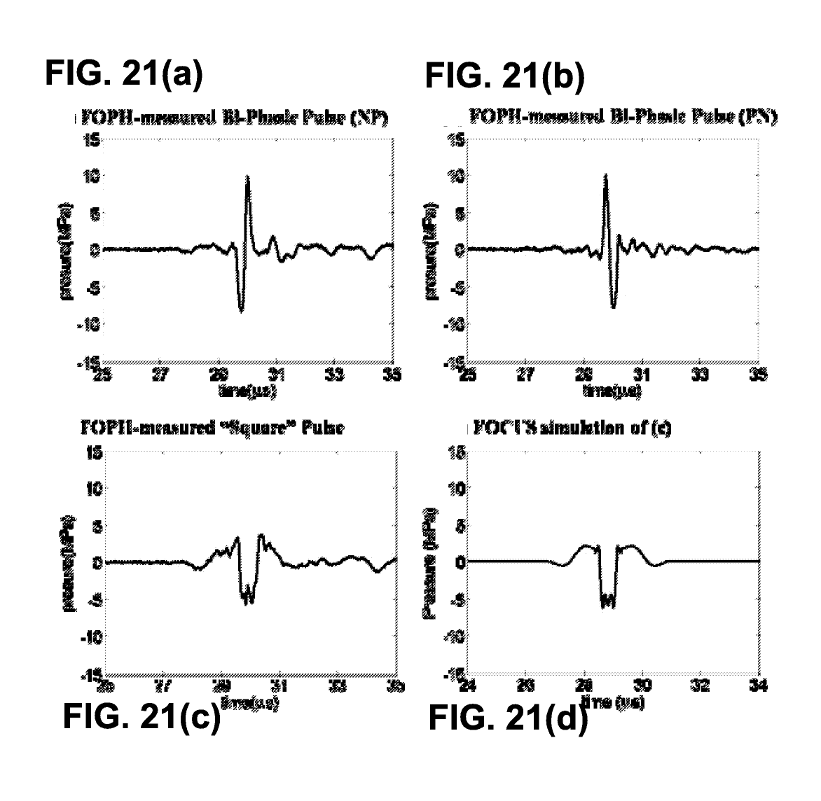

In this disclosure, the time delays and excitation amplitudes for each element can be specifically selected to generate monopolar pulses; however, a different set of time delays and excitation amplitudes can be chosen to synthesize different types of waveforms. FIG. 21 demonstrates some example waveforms that can be generated by a multi-element frequency-compounding transducer. FIG. 21(a) shows an FOPH-measured bi-phasic pulse with a negative-polarity pulse followed by a positive-polarity pulse (an “NP” pulse), and FIG. 21(b) shows an FOPH-measured bi-phasic pulse with a positive-polarity pulse followed by a negative-polarity pulse (a “PN” pulse). FIG. 21(c) shows an FOPH-measured “square” pulse, and FIG. 21(d) is a simulation for FIG. 21(c) using the FOCUS simulation tool. Although this “square” pulse generated by the transducer does not resemble an ideal square pulse, the shape can improve considerably with a transducer using a large number of elements including a much wider variety of frequencies.

As demonstrated in FIG. 21, this frequency compounding technique for monopolar pulse generation can be generalized to a broader concept, namely, “waveform synthesis” using frequency compounding. With a sufficient number of elements and a wide variety of frequencies, a frequency-compounding transducer can become an arbitrary waveform synthesizer by appropriately adjusting time delays and excitation amplitudes to individual elements. Moreover, optimization algorithms can allow more precise choice of time delays, frequencies, and array geometries for synthesis of waveforms needed for unique applications.

This disclosure demonstrates the feasibility of generating monopolar pulses using a multi-element frequency-compounding transducer. By adjusting time delays for individual frequency components and allowing their principal peak negatives to arrive at the focus of the transducer concurrently, monopolar pulses with a dominant negative phase (negative-polarity pulses) could be generated. By inverting the excitation pulses to individual elements, monopolar pulses with a dominant positive phase (positive-polarity pulses) could also be generated. Negative-polarity pulses with combined P− higher than the intrinsic threshold were capable of creating cavitational lesion-producing bubble clouds, and the size of corresponding lesions in RBC phantoms increased with increased combined P−. Neither cavitational bubble clouds nor lesions were generated in RBC phantoms using positive-polarity pulses with combined P+ similar to the level of combined P− used in RBC phantom experiments with negative-polarity pulses. Therefore, Frequency Compounding allows the generation of highly functional Histotripsy pulses with no extraneous complicating features.

These frequency-compounded monopolar pulses can have many applications in both ultrasonic imaging and therapy. Non-coherent excitation pulses, for example, would minimize speckle in ultrasound images. Moreover, a frequency-compounding transducer can potentially become an arbitrary waveform synthesizer given that the transducer has a sufficient number of elements with a wide variety of resonant frequencies.

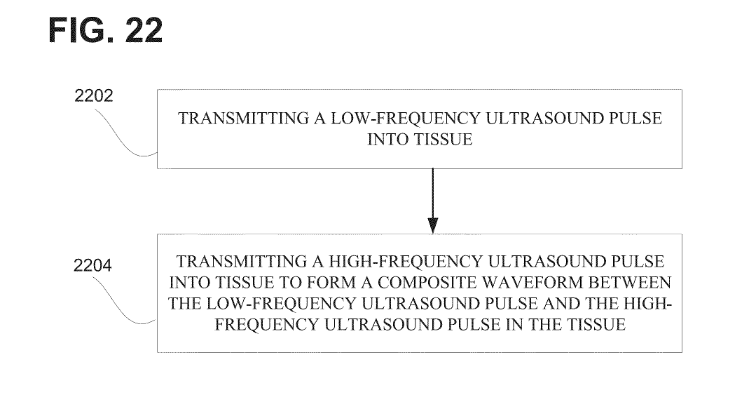

Methods of treatment and use are also provided. In one embodiment, referring to FIG. 22 a method of providing ultrasound therapy to tissue comprises step 2202, transmitting a low-frequency ultrasound pulse into tissue, and, step 2204 transmitting a high-frequency ultrasound pulse into tissue to form a composite waveform between the low-frequency ultrasound pulse and the high-frequency ultrasound pulse in the tissue. In some embodiments, a time delay between transmission of the low-frequency ultrasound pulse and transmission of the high-frequency ultrasound pulse can be selected such that a peak negative pressure value of the composite waveform exceeds an intrinsic threshold for cavitation in the tissue. In other embodiments, the time delay between pulses can be adjusted in real-time to achieve the peak negative pressure value that exceeds the intrinsic threshold.

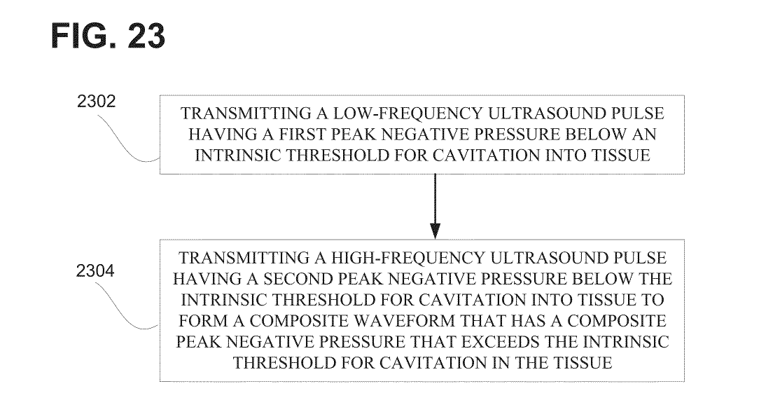

Another method of providing ultrasound therapy to tissue is also provided, as shown in FIG. 23, comprising step 2302, transmitting a low-frequency ultrasound pulse into tissue, wherein the low-frequency ultrasound pulse has a first peak negative pressure below an intrinsic threshold for cavitation in the tissue, and step 2304 transmitting a high-frequency ultrasound pulse into tissue to form a composite waveform between the low-frequency ultrasound pulse and the high-frequency ultrasound pulse, wherein the high-frequency ultrasound pulse has a second peak negative pressure below the intrinsic threshold for cavitation in the tissue, wherein a composite peak negative pressure of the composite waveform exceeds the intrinsic threshold for cavitation in the tissue. In some embodiments, a time delay between transmission of the low-frequency ultrasound pulse and transmission of the high-frequency ultrasound pulse can be selected such that a peak negative pressure value of the composite waveform exceeds an intrinsic threshold for cavitation in the tissue. In other embodiments, the time delay between pulses can be adjusted in real-time to achieve the peak negative pressure value that exceeds the intrinsic threshold.

In some embodiments, the high-frequency ultrasound pulse is transmitted at a time delay with respect to the low-frequency ultrasound pulse to allow for a maximum peak negative pressure value to be achieved with the composite waveform.

In other embodiments, the low-frequency ultrasound pulse ranges from 100 kHz and 1 MHz and the high-frequency ultrasound pulse ranges from 2 MHz to 10 MHz.

In some embodiments, the low-frequency ultrasound pulse and the high-frequency ultrasound pulse are transmitted with a single transducer.

In one embodiment, the low-frequency ultrasound pulse and the high-frequency ultrasound pulse are transmitted with more than one transducer.

In some embodiments, the method further comprises adjusting a proportion of an amplitude of the low-frequency ultrasound pulse relative to an amplitude of the high-frequency ultrasound pulse to adjust a size of a lesion created in the tissue. In one embodiment, increasing the proportion of the amplitude of the high-frequency ultrasound pulses decreases a size of the lesion.

In some embodiments, the low-frequency ultrasound pulse and the high-frequency ultrasound pulse are transmitted simultaneously and combine constructively to form the composite waveform.

In one embodiment, the time delay comprises 0 μs such that the peak negative pressure value comprises a maximum peak negative pressure value.

In some embodiments, the ultrasound pulses comprise short (<20 μsec), high pressure (peak negative pressure >10 MPa) shockwave ultrasound pulses at a low duty cycle, typically <5%, minimizing thermal effects.

In one embodiment, the composite waveform creates cavitation in the tissue to form a lesion in the tissue.

In another embodiment, the transmitting the low-frequency ultrasound pulse step further comprises transmitting the low-frequency ultrasound pulse with an electronic controller coupled to an ultrasound transducer. In further embodiments, the transmitting the high-frequency ultrasound pulse step further comprises transmitting the high-frequency ultrasound pulse with an electronic controller coupled to an ultrasound transducer.

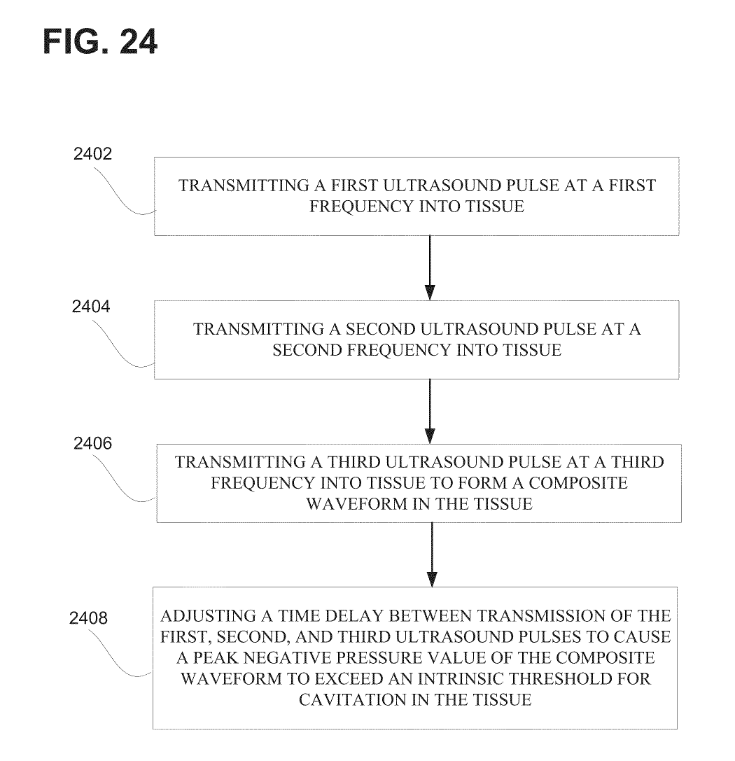

Referring to FIG. 24, another method of providing ultrasound therapy to tissue is further provided, comprising step 2402, transmitting a first ultrasound pulse at a first frequency into tissue, step 2404, transmitting a second ultrasound pulse at a second frequency into tissue, step 2406, transmitting a third ultrasound pulse at a third frequency into tissue, wherein the first, second, and third ultrasound pulses combine to form a composite waveform in the tissue, and step 2408, adjusting a time delay between transmission of the first, second, and third ultrasound pulses to cause a peak negative pressure value of the composite waveform to exceed an intrinsic threshold for cavitation in the tissue.

In some embodiments, the ultrasound pulses are transmitted with a single transducer.

In other embodiments, the ultrasound pulses are transmitted with a multi-element transducer.

In some embodiments, the method further comprises adjusting a proportion of an amplitude of the first, second, and third ultrasound pulses to adjust a size of a lesion created in the tissue.

In one embodiment, the first, second, and third ultrasound pulses are transmitted simultaneously and combine constructively to form the composite waveform.

In some embodiments, the time delay comprises 0 μs, and wherein the peak negative pressure value comprises a maximum peak negative pressure value.

In one embodiment, the ultrasound pulses comprise short (<20 μsec), high pressure (peak negative pressure >10 MPa) shockwave ultrasound pulses at a low duty cycle, typically <5%, minimizing thermal effects.

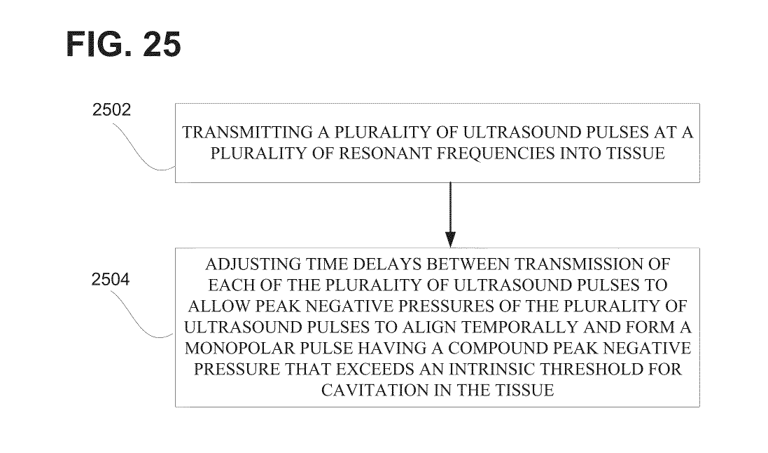

Referring to FIG. 25, a method of providing ultrasound therapy to tissue is provided, comprising step 2502, transmitting a plurality of ultrasound pulses at a plurality of resonant frequencies into tissue, and step 2504, adjusting time delays between transmission of each of the plurality of ultrasound pulses to allow peak negative pressures of the plurality of ultrasound pulses to align temporally and form a monopolar pulse having a compound peak negative pressure that exceeds an intrinsic threshold for cavitation in the tissue.

In one embodiment, the method further comprises creating cavitation in the tissue with the monopolar pulse to form a lesion in the tissue.

In some embodiments, the plurality of resonant frequencies range from 100 kHz to 10 MHz.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of providing ultrasound therapy to tissue, comprising the steps of:
transmitting a low-frequency ultrasound pulse into tissue; and
transmitting a high-frequency ultrasound pulse into tissue to form a composite waveform between the low-frequency ultrasound pulse and the high-frequency ultrasound pulse in the tissue, wherein a peak negative pressure value of the composite waveform exceeds an intrinsic threshold for cavitation in the tissue.

2. The method of claim 1 wherein the high-frequency ultrasound pulse is transmitted at a time delay with respect to the low-frequency ultrasound pulse to allow for a maximum peak negative pressure value to be achieved with the composite waveform.

3. The method of claim 1 wherein the low-frequency ultrasound pulse ranges from 100 kHz and 1 MHz and the high-frequency ultrasound pulse ranges from 2 MHz to 10 MHz.

4. The method of claim 1 wherein the low-frequency ultrasound pulse and the high-frequency ultrasound pulse are transmitted with a single transducer.

5. The method of claim 1 wherein the low-frequency ultrasound pulse and the high-frequency ultrasound pulse are transmitted with more than one transducer.

6. The method of claim 1 further comprising adjusting a proportion of an amplitude of the low-frequency ultrasound pulse relative to an amplitude of the high-frequency ultrasound pulse to adjust a size of a lesion created in the tissue.

7. The method of claim 4, wherein increasing the proportion of the amplitude of the high-frequency ultrasound pulses decreases a size of the lesion.

8. The method of claim 1 wherein the low-frequency ultrasound pulse and the high-frequency ultrasound pulse are transmitted simultaneously and combine constructively to form the composite waveform.

9. The method of claim 1 wherein the time delay comprises 0 μs such that the peak negative pressure value comprises a maximum peak negative pressure value.

10. The method of claim 1 wherein the ultrasound pulses comprise short (<20 μsec), high pressure (peak negative pressure >10 MPa) shockwave ultrasound pulses at a low duty cycle, typically <5%, minimizing thermal effects.

11. The method of claim 1, wherein the composite waveform creates cavitation in the tissue to form a lesion in the tissue.

12. The method of claim 1, wherein the transmitting the low-frequency ultrasound pulse step further comprises transmitting the low-frequency ultrasound pulse with an electronic controller coupled to an ultrasound transducer.

13. The method of claim 1, wherein the transmitting the high-frequency ultrasound pulse step further comprises transmitting the high-frequency ultrasound pulse with an electronic controller coupled to an ultrasound transducer.

14. A method of providing ultrasound therapy to tissue, comprising the steps of:
transmitting a low-frequency ultrasound pulse into tissue, wherein the low-frequency ultrasound pulse has a first peak negative pressure below an intrinsic threshold for cavitation in the tissue; and
transmitting a high-frequency ultrasound pulse into tissue to form a composite waveform between the low-frequency ultrasound pulse and the high-frequency ultrasound pulse, wherein the high-frequency ultrasound pulse has a second peak negative pressure below the intrinsic threshold for cavitation in the tissue, wherein a composite peak negative pressure of the composite waveform exceeds the intrinsic threshold for cavitation in the tissue.

15. The method of claim 15 wherein the high-frequency ultrasound pulse is transmitted at a time delay with respect to the low-frequency ultrasound pulse to allow for a maximum peak negative pressure value to be achieved with the composite waveform.

16. The method of claim 14 wherein the low-frequency ultrasound pulse ranges from 100 kHz and 1 MHz and the high-frequency ultrasound pulse ranges from 2 MHz to 10 MHz.

17. The method of claim 14, wherein the composite waveform creates cavitation in the tissue to form a lesion in the tissue.

18. The method of claim 14 wherein the low-frequency ultrasound pulse and the high-frequency ultrasound pulse are transmitted with a single transducer.

19. The method of claim 14 wherein the low-frequency ultrasound pulse and the high-frequency ultrasound pulse are transmitted with more than one transducer.

20. The method of claim 14 further comprising adjusting a proportion of an amplitude of the low-frequency ultrasound pulse relative to an amplitude of the high-frequency ultrasound pulse to adjust a size of a lesion created in the tissue.

21. The method of claim 20, wherein increasing the proportion of the amplitude of the high-frequency ultrasound pulses decreases a size of the lesion.

22. The method of claim 14 wherein the low-frequency ultrasound pulse and the high-frequency ultrasound pulse are transmitted simultaneously and combine constructively to form the composite waveform.

23. The method of claim 14 wherein the time delay comprises 0 μs such that the composite peak negative pressure comprises a maximum peak negative pressure value.

24. The method of claim 14 wherein the ultrasound pulses comprise short (<20 μsec), high pressure (peak negative pressure >10 MPa) shockwave ultrasound pulses at a low duty cycle, typically <5%, minimizing thermal effects.

25. The method of claim 14, wherein the transmitting the low-frequency ultrasound pulse step further comprises transmitting the low-frequency ultrasound pulse with an electronic controller coupled to an ultrasound transducer.

26. The method of claim 14, wherein the transmitting the high-frequency ultrasound pulse step further comprises transmitting the high-frequency ultrasound pulse with an electronic controller coupled to an ultrasound transducer.

27. A Histotripsy therapy system, comprising:
a pulse generator;
an ultrasound therapy transducer coupled to the pulse generator and having a plurality of transducer elements, wherein one or more transducer elements of the ultrasound therapy transducer are configured to transmit a low-frequency ultrasound pulse into tissue that has a first peak negative pressure below an intrinsic threshold for cavitation in the tissue, and wherein one or more transducer element of the ultrasound therapy transducer are configured to transmit a high-frequency ultrasound pulse into tissue that has a second peak negative pressure below the intrinsic threshold for cavitation in the tissue; and
an electronic controller coupled to the pulse generator and the ultrasound therapy transducer, the electronic controller configured to control transmission of the high-frequency ultrasound pulse relative to transmission of the low-frequency ultrasound pulse to form a composite waveform between the low-frequency ultrasound pulse and the high-frequency ultrasound pulse that has a composite peak negative pressure that exceeds the intrinsic threshold for cavitation in the tissue.

28. A method of providing ultrasound therapy to tissue, comprising the steps of:
transmitting a first ultrasound pulse at a first frequency into tissue;
transmitting a second ultrasound pulse at a second frequency into tissue;
transmitting a third ultrasound pulse at a third frequency into tissue, wherein the first, second, and third ultrasound pulses combine to form a composite waveform in the tissue; and
adjusting a time delay between transmission of the first, second, and third ultrasound pulses to cause a peak negative pressure value of the composite waveform to exceed an intrinsic threshold for cavitation in the tissue.

29. The method of claim 20 wherein the ultrasound pulses are transmitted with a single transducer.

30. The method of claim 20 wherein the ultrasound pulses are transmitted with a multi-element transducer.

31. The method of claim 20 further comprising adjusting a proportion of an amplitude of the first, second, and third ultrasound pulses to adjust a size of a lesion created in the tissue.

32. The method of claim 20 wherein the first, second, and third ultrasound pulses are transmitted simultaneously and combine constructively to form the composite waveform.

33. The method of claim 20 wherein the time delay comprises 0 μs, and wherein the peak negative pressure value comprises a maximum peak negative pressure value.

34. The method of claim 20 wherein the ultrasound pulses comprise short (<20 μsec), high pressure (peak negative pressure >10 MPa) shockwave ultrasound pulses at a low duty cycle, typically <5%, minimizing thermal effects.

35. A method of providing ultrasound therapy to tissue, comprising the steps of:
transmitting a plurality of ultrasound pulses at a plurality of resonant frequencies into tissue; and
adjusting time delays between transmission of each of the plurality of ultrasound pulses to allow peak negative pressures of the plurality of ultrasound pulses to align temporally and form a monopolar pulse having a compound peak negative pressure that exceeds an intrinsic threshold for cavitation in the tissue.

36. The method of claim 35 further comprising creating cavitation in the tissue with the monopolar pulse to form a lesion in the tissue.

37. The method of claim 35 wherein the plurality of resonant frequencies range from 100 kHz to 10 MHz.

38. A Histotripsy therapy system, comprising:
a pulse generator;
an ultrasound therapy transducer coupled to the pulse generator and having a plurality of transducer elements, wherein one or more transducer elements of the ultrasound therapy transducer are configured to transmit a plurality of ultrasound pulses into tissue at a plurality of resonant frequencies; and
an electronic controller coupled to the pulse generator and the ultrasound therapy transducer, the electronic controller configured to adjust time delays between transmission of each of the plurality of ultrasound pulses to allow peak negative pressures of the plurality of ultrasound pulses to align temporally and form a monopolar pulse having a compound peak negative pressure that exceeds an intrinsic threshold for cavitation in the tissue.
